(19) United States
(12) Patent Application Publication
(10) Pub. No.: US 2026/0357175 A1
(43) Pub. Date: Nov. 12, 2026

(54) Microfluidic Platform Modulated By Dual MicroTesla Pumps For Cell Co-Culturing In Diabetes And Hypoxia

(71) Applicant: The Regents of The University of Michigan, Ann Arbor, MI (US)

(72) Inventors: Mohamad ORABI, Inkster, MI (US); Kai DUAN, Troy, MI (US); Tae-Hwa CHUN, Ann Arbor, MI (US); Joe F. LO, Dearborn, MI (US)

(73) Assignee: The Regents of The University of Michigan, Ann Arbor, MI (US)

(21) Appl. No.: 19/672,045

(22) Filed: May 8, 2026

Related U.S. Application Data
(60) Provisional application No. 63/801,956, filed on May 8, 2025.

(51) Int. Cl. C12M 3/06; C12M 1/00; C12M 1/04; C12M 1/34; C12M 1/42

(52) U.S. Cl. CPC C12M 23/16; C12M 23/24; C12M 29/00; C12M 29/10; C12M 35/04; C12M 41/34

(57) ABSTRACT

A microfluidic device for regulating flow shear stress on cells includes two pairs of enclosed perfusion chambers, one with a first and a second perfusion chamber and another with a third and a fourth perfusion chamber. Each of the perfusion chambers comprises a first inlet for receiving cells, a second inlet for receiving a fluid medium, a first outlet for removing cells, and a second outlet where fluid medium exits. A gas-permeable membrane defines a side of each of the perfusion chambers. First and second microTesla pumps are included. A microfluidic gas channel in fluid communication with and disposed along the gas permeable membrane opposite to the first surface. An inlet of the microfluidic gas channel receives the gas that transfers through select regions of the gas permeable membrane into each of the first, second, third, and fourth perfusion chambers. Methods for co-culturing distinct cells are also provided.

### GOVERNMENT SUPPORT

This invention was made with government support under 1751426 awarded by the National Science Foundation. The government has certain rights in the invention.

### CROSS-REFERENCE TO RELATED APPLICATIONS

This application claims the benefit of U.S. Provisional Application No. 63/801,956, filed May 8, 2025. The entire disclosure of the above application is incorporated herein by reference.

### FIELD

This disclosure relates to a microfluidic device for regulating flow shear stress (FSS) and an oxygen environment for cell culture studies under diabetic and hypoxic conditions, where the microfluidic device comprises dual microTesla pumps and methods for co-culturing distinct cell types in a microfluidic device under flow shear stress.

### BACKGROUND

This section provides background information related to the present disclosure which is not necessarily prior art.

Oxygen control plays an important role for biomedical research, particularly in the field of microfluidics. Oxygen is a key regulator of cellular function, influencing processes such as cell metabolism, differentiation, and proliferation. In microfluidic systems, where cells are cultured in controlled environments that mimic physiological conditions, precise regulation of oxygen levels is essential for studying cellular responses under hypoxic (low oxygen) or normoxic (normal oxygen) conditions. These systems enable researchers to investigate disease models, drug responses, and tissue engineering in a more realistic and scalable way. Advancements in microfluidic technology have led to the development of devices capable of modulating oxygen gradients and flow dynamics, offering new insights into how oxygen affects biological systems at the microscale. The integration of oxygen control into microfluidic platforms is a growing area of interest, as it provides a more accurate representation of in vivo conditions, thereby enhancing the reliability and applicability of research outcomes in areas like cancer biology, stem cell research, and regenerative medicine.

Further, shear stress in microfluidics is also important for mimicking the mechanical forces experienced by cells in physiological environments, such as blood vessels. Shear stress is a force generated by the blood flow on blood vessels, more specifically on the inner layer or endothelium of the blood vessel. Blood flow patterns can change endothelial cell (EC) activities. For example, pulsatile or steady laminar flow can stimulate ECs to secrete functional factors and suppress coagulation promoting cell survival. However, when endothelial cells experience chronic arrhythmic changes in hemodynamic forces, they may exhibit different behavior that enhances cellular turnover (proliferation and apoptosis) and facilitate adhesion of monocytes onto endothelium, suggesting a higher risk of function loss and atherosclerotic plaque formation. Simulating shear stress conditions with flowing culture medium in vitro can help to stimulate endothelial cells in controlled ways. Thus, it is desirable to develop microfluidic devices, including organ-on-chip (OOC) technology, which can provide the capability of regulating flow shear stress on cells being cultured, while also controlling the environment surrounding such cells by modulating oxygen and other gas levels.

### SUMMARY

This section provides a general summary of the disclosure and is not a comprehensive disclosure of its full scope or all of its features.

In certain aspects, the present disclosure relates to a microfluidic device for regulating flow shear stress on cells. The microfluidic device comprising a first pair of enclosed perfusion chambers comprising a first perfusion chamber and a second perfusion chamber fluidly coupled with one another. The microfluidic device also comprises a second pair of enclosed perfusion chambers comprising a third perfusion chamber and a fourth perfusion chamber fluidly coupled with one another. Each of the first, second, third, and fourth perfusion chambers comprises a first inlet for receiving a stream comprising cells, a second inlet for receiving a fluid medium, a first outlet for removing the stream comprising cells, a second outlet through which the fluid medium exits. The microfluidic device includes a gas permeable membrane having a first surface that defines at least one side of each of the first, second, third, and fourth perfusion chambers. The microfluidic device further includes a first microTesla pump in fluid communication with the second inlet of the first perfusion chamber and the second inlet of the second perfusion chamber configured to circulate the fluid medium therethrough. The microfluidic device also includes a second microTesla pump in fluid communication with the second inlet of the third perfusion chamber and the second inlet of the fourth perfusion chamber configured to circulate the fluid medium therethrough. A microfluidic gas channel is in fluid communication with and disposed along a second surface of the gas permeable membrane opposite to the first surface. An inlet of the microfluidic gas channel receives gas that transfers through select regions of the gas permeable membrane into each of the first, second, third, and fourth perfusion chambers.

In one aspect, the first perfusion chamber contains a first type of cells and the second perfusion chamber contains a second type of cells distinct from the first type. Further, the third perfusion chamber contains the first type of cells, and the fourth perfusion chamber contains the second type of cells.

In one further aspect, the first type of cells and the second type of cells are independently selected from pancreatic beta cells and adipocytes.

In one aspect, the microfluidic gas channel has a height of greater than or equal to about 0.1 millimeters to less than or equal to about 2 millimeters.

In one aspect, the gas permeable membrane has a thickness of greater than or equal to about 50 micrometers to less than or equal to about 250 micrometers.

In one aspect, the gas permeable membrane comprises a porous polydimethylsiloxane (PDMS).

In one aspect, the first inlet and the first outlet are configured to be sealed.

In one aspect, each of the first, second, third, and fourth perfusion chambers has a height of greater than or equal to about 2 millimeters to less than or equal to about 4 millimeters.

In one aspect, the fluid medium continuously flows through the first, second, third, and fourth perfusion chambers during a second operational mode.

In one aspect, the gas comprises oxygen and an oxygen level in each of the first, second, third, and fourth perfusion chambers is independently controlled to be at least one of a hypoxic environment or a normoxic environment.

In one aspect, the first microTesla pump and the first pair of enclosed perfusion chambers define a first recirculation loop, where the fluid medium exits the first microTesla, is pumped into the second inlet of the first perfusion chamber, exits the second outlet of the first perfusion chamber, enters the second outlet of the second perfusion chamber, exits the second outlet of the second perfusion chamber, and enters the first microTesla pump. Further, the second microTesla pump and the second pair of enclosed perfusion chambers define a second recirculation loop, where the fluid medium exits the second microTesla pump, is pumped into the second inlet of the third perfusion chamber, exits the second outlet of the third perfusion chamber, enters the second outlet of the fourth perfusion chamber, exits the second outlet of the fourth perfusion chamber, and enters the second microTesla pump.

In one aspect, the cells grow on the first surface of the gas permeable membrane in each of the first, second, third, and fourth perfusion chambers.

In certain further aspects, the present disclosure relates to a method for co-culturing distinct cell types in a microfluidic device under flow shear stress. The method comprises pumping a first fluid medium in a first microfluidic recirculation loop comprising a first pair of enclosed perfusion chambers that includes a first perfusion chamber comprising a first type of cells and a second perfusion chamber comprising a second type of cells and a first microTesla pump to induce flow shear stress conditions on the first type of cells in the first perfusion chamber and the second type of cells in the second perfusion chamber. The method also comprises pumping a second fluid medium in a second microfluidic recirculation loop comprising a second pair of enclosed perfusion chambers that include a third perfusion chamber comprising the first type of cells and a fourth perfusion chamber comprising the second type of cells, and a second microTesla pump to induce flow shear stress conditions on the first type of cells in the third perfusion chamber and the second type of cells in the fourth perfusion chamber. Further, the method comprises introducing gas into a microfluidic gas channel where the gas transfers into each of the first, second, third, and fourth perfusion chambers through a gas permeable membrane, where the gas permeable membrane defines at least one wall of the microfluidic chamber.

In one aspect, the first fluid medium continuously flows through the first and second perfusion chambers during a first operational mode and the second fluid medium continuously flows through the third and fourth perfusion chambers during the first operational mode.

In one aspect, the pumping of the first fluid, pumping of the second fluid, and the introducing occur concurrently.

In one aspect, each of the first, second, third, and fourth perfusion chambers comprises a first inlet for receiving a stream comprising either the first type of cells or the second type of cells. A second inlet for each of the first, second, third, and fourth perfusion chambers receives either the first fluid medium or the second fluid medium, a first outlet for removing the stream, and a second outlet through which the first fluid medium or the second fluid medium exits.

In one aspect, the stream comprises the first type of cells and the method further comprises passing the stream through the first perfusion chamber and the third perfusion chamber to introduce the first type of cells. After passing the stream through the first perfusion chamber and the third perfusion chamber, the method includes sealing the first outlet of the first perfusion chamber and the first outlet of the third perfusion chamber.

In one aspect, the stream comprises the second type of cells. The method further comprises passing the stream through the second perfusion chamber and the fourth perfusion chamber to introduce the second type of cells, and after passing the stream through the second perfusion chamber and the fourth perfusion chamber, sealing the first outlet of the second perfusion chamber and the first outlet of the fourth perfusion chamber.

In one aspect, the first fluid medium passes through the first microfluidic recirculation loop at a flow rate of greater than or equal to about 0.62 mL/minute to less than or equal to about 1 mL/minute and the second fluid medium passes through the second microfluidic recirculation loop at a flow rate of greater than or equal to about 0.62 mL/minute to less than or equal to about 1 mL/minute.

In one aspect, the method further comprises assessing interactions of the first cell type and the second cell type under an influence of shear stress.

In one aspect, the assessing interactions of the first cell type and the second cell type under an influence of shear stress is related to at least two distinct diseases or conditions.

In one aspect, the gas comprises oxygen and is transferred through the gas permeable membrane into each of the first, second, third, and fourth perfusion chambers and an oxygen level in each of the first, second, third, and fourth perfusion chambers is independently controlled to be at least one of a hypoxic environment or a normoxic environment.

In one aspect, the first type of cells and the second type of cells are independently selected from pancreatic beta cells and adipocytes.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIGS. 1A–1C. FIG. 1A is an exploded assembly perspective view of a microfluidic device prepared in accordance with certain aspects of the present disclosure. FIG. 1B shows a top view of the microfluidic device. FIG. 1C shows components, including rotors and housing, of a microTesla pump according to certain aspects of the present disclosure.

FIGS. 2A–2D show an example of a method for forming a shear force flow (SFF)–enabled microfluidic device according to certain aspects of the present disclosure. FIG. 2A shows a master template being fabricated using Fused Deposition Modeling (FDM) printing, over which a polymer like polydimethylsiloxane (PDMS) can be poured and cured/crosslinked. Once cured, the PDMS layer was peeled off and bonded to a glass substrate. FIG. 2B shows on the left side of an arrow, a microfluidic device design created using Autodesk Inventor software, while on the right side of the arrow, the microfluidic device manufactured using the FDM method is shown. FIG. 2C shows microTesla pump components and assembly for integration into the device. FIG. 2D shows images illustrating injection of a cell culture medium into the embedded microTesla pumps followed by media flow over the cultured cells in accordance with certain aspects of the present disclosure.

FIGS. 3A–3B. FIG. 3A illustrates a side sectional view of another variation of a microfluidic device according to certain aspects of the present disclosure. FIG. 3B illustrates a system into which the microfluidic device of FIG. 3A may be incorporated.

FIG. 4 is a chart illustrating the simultaneous control of oxygen, shear flow, and glucose stimulation at the cells cultured inside such a microfluidic device prepared in accordance with certain aspects of the present disclosure.

FIGS. 5A–5B. FIG. 5A illustrates oxygen concentration (%) as a function of time (minutes) for a microfluidic device prepared according to certain aspects of the present disclosure. FIG. 5B illustrates a percent change in oxygen concentration as a function of time for the microfluidic device.

FIGS. 6A–6F. FIG. 6A shows fluorescence images for adipocytes and INS1 cells in monoculture without shear (MC), monoculture with shear (MCS), and co-culture (CC) environments. FIG. 6B shows cell viability quantification of the images shown in FIG. 6A. FIG. 6C shows cell viability of adipocytes over 3 days highlighting the importance of co-culture environment and shear stress impact on monoculture cells. FIG. 6D shows live and dead cell count for adipocytes over 3 days to analyze cell viability in FIG. 6C. FIG. 6E shows the cell viability for INS1 cells over 3 days highlighting the importance of co-culture environment and shear stress impact on monoculture cells. FIG. 6F shows live and dead cell count for INS1 cells over 3 days to analyze cell viability in FIG. 6E.

FIGS. 7A–7C. FIG. 7A shows the differentiation percentage of 3T3-L1 cells into adipocytes under shear stress and co-culture (in monoculture without shear (MC), monoculture with shear (MCS), and co-culture (CC) environments). FIG. 7B shows lipid droplet size highlighting the impact of shear stress on monoculture cells and the co-culture environment. FIG. 7C shows the bright field and Oil Red O staining images for lipid droplets of adipocytes.

FIGS. 8A–8E. FIG. 8A shows fluorescence images of mitochondrial ATP and reactive oxygen species (ROS) for adipocytes and INS1 cells in monoculture without shear (MC), monoculture with shear (MCS), and co-culture (CC) environments. FIG. 8B shows the normalized mitochondrial ATP intensity for adipocytes over 24 hrs. (1 day). FIG. 8C shows the normalized reactive oxygen species intensity for adipocytes over 24 hrs. FIG. 8D shows the normalized mitochondrial ATP intensity for INS1 cells over 24 hrs. FIG. 8E shows the normalized reactive oxygen species intensity for INS1 cells over 24 hrs.

FIGS. 9A–9F. FIG. 9A shows the cell viability of co-cultured adipocytes on-chip under normoxia and hypoxia for 3 days. FIG. 9B shows normalized mitochondrial ATP intensity of adipocytes under the same period and conditions as FIG. 9A. FIG. 9C shows normalized ROS intensity of adipocytes under the same period and conditions as in FIGS. 9A and 9B. FIG. 9D shows the cell viability of co-cultured INS1 cells on-chip under normoxia and hypoxia for 3 days. FIG. 9E shows the normalized mitochondrial ATP intensity of INS1 cells under the same period and conditions as in FIG. 9D. FIG. 9F shows normalized ROS intensity of INS1 cells under the same period and conditions as in FIGS. 9D and 9E.

FIGS. 10A–10E. FIG. 10A shows impact of insulin modulations on IL-6 secretion from monoculture adipocytes under normoxia and hypoxia. FIG. 10B shows impact of insulin modulations on TNF-α secretion from monoculture adipocytes under the same conditions as in FIG. 10A. FIG. 10C shows impact of insulin modulations on adiponectin secretion from monoculture adipocytes under the same conditions as in FIGS. 10A–10B. FIG. 10D shows the impact of IL-6 modulation on calcium (FURA 2-A.M.) secretion from monoculture INS1 cells under normoxia and hypoxia. FIG. 10E shows impact of IL-6 modulation on insulin secretion from monoculture INS1 cells under the same conditions as in FIG. 10D.

FIGS. 11A–11E. FIG. 11A shows impact of glucose modulation on IL-6 secretion from co-cultured adipocytes under normoxia and hypoxia. FIG. 11B shows impact of glucose modulation on TNF-α secretion from co-cultured adipocytes under the same conditions as in FIG. 11A. FIG. 11C shows impact of glucose modulation on adiponectin secretion from co-cultured adipocytes under the same conditions as in FIGS. 11A and 11B. FIG. 11D shows impact of glucose modulation on calcium secretion from co-cultured INS1 cells under normoxia and hypoxia. FIG. 11E shows the impact of glucose modulation on insulin secretion from co-cultured INS1 cells under the same conditions as FIG. 11D.

FIGS. 12A–12B. FIG. 12A shows the normoxic and hypoxic impact on IL-6, TNF-α, and adiponectin secretion from co-cultured adipocytes and calcium and insulin secretion from co-cultured INS1 cells. FIG. 12B shows the impact of 5.5 mM and 25 mM glucose on IL-6, TNF-α, and adiponectin secretion from co-cultured adipocytes and calcium and insulin secretion from co-cultured INS1 cells.

FIGS. 13A–13I show IL-6, TNF-α, and adiponectin secretion from co-cultured adipocytes when exposed to glucose modulations under hypoxia conditions. FIGS. 13A, 13B and 13C show IL-6 concentrations over 3 days under 5.5 mM glucose (FIG. 13A), 12.5 mM glucose (FIG. 13B), and 25 mM glucose (FIG. 13C). FIGS. 13D, 13E and 13F show TNF-α concentrations over 3 days under 5.5 mM glucose (FIG. 13D), 12.5 mM glucose (FIG. 13E), and 25 mM glucose (FIG. 13F) concentrations. FIGS. 13G, 13H and 13I show adiponectin concentrations over 3 days under 5.5 mM glucose (FIG. 13G), 12.5 mM glucose (FIG. 13H), and 25 mM glucose (FIG. 13I) concentrations.

FIGS. 14A–14F show calcium influx detection (by FURA 2) and insulin concentration from co-cultured beta INS1 cells when exposed to glucose modulations under hypoxia conditions. FIGS. 14A, 14B and 14C, show calcium FURA 2 response over 3 days under 5.5 mM glucose (FIG. 14A), 12.5 mM glucose (FIG. 14B), and 25 mM glucose (FIG. 14C). FIGS. 14D, 14E and 14F, show insulin concentrations over 3 days under 5.5 mM glucose (FIG. 14D), 12.5 mM glucose (FIG. 14E), and 25 mM glucose (FIG. 14F) concentrations.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

In various aspects, the present disclosure contemplates a microfluidic device for cell culture that enables regulating flow shear stress on cells. Further, the microfluidic device can control environmental conditions in perfusion chambers, such as controlling oxygen content. For example, the present disclosure contemplates various microfluidic devices designed to modulate shear stress and oxygen levels in cell culture studies. These devices incorporate a micropump, such as microTesla (µTesla) pumps, enabling precise flow rate control and facilitating the simulation of blood flow in systems like the pancreas.

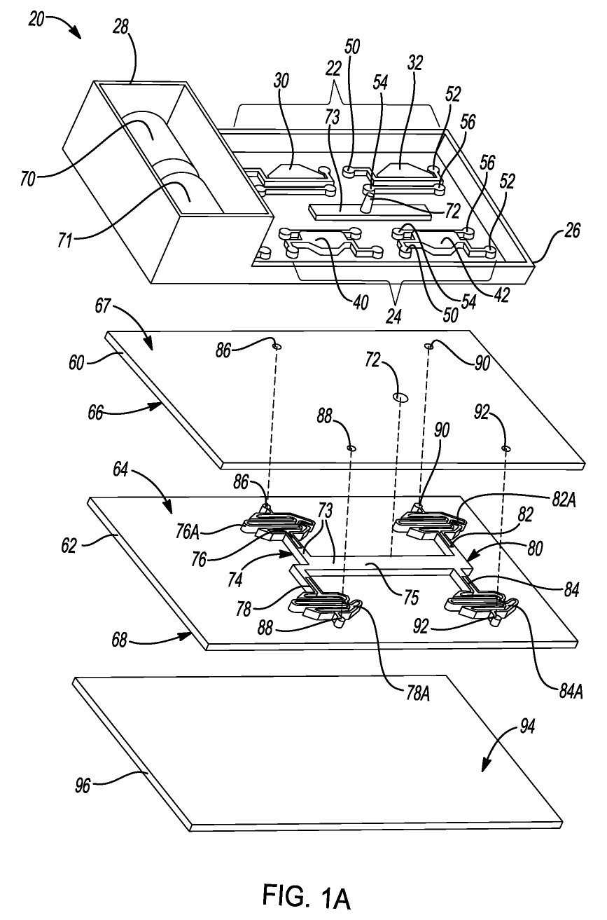

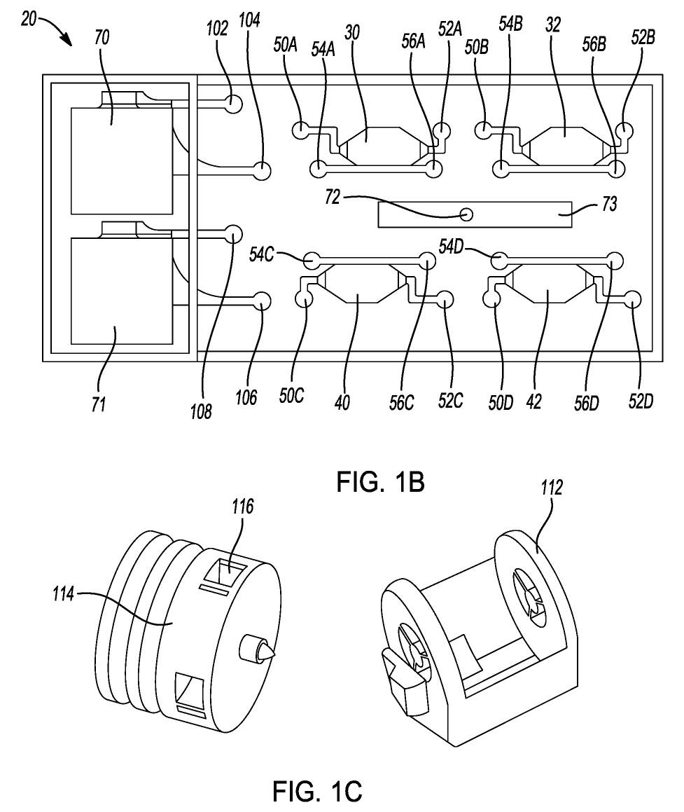

By way of example, FIGS. 1A–1D, show a microfluidic device 20 that includes both a first pair of enclosed perfusion chambers 22 and a second pair of enclosed perfusion chambers 24. A solid cover component 26 defines a housing 28 or enclosure for pumps. The solid cover component 26 also defines the first pair of enclosed perfusion chambers 22 that includes a first perfusion chamber 30 and a second perfusion chamber 32 fluidly coupled with one another. The second pair of enclosed perfusion chambers 24 includes a third perfusion chamber 40 and a fourth perfusion chamber 42 fluidly coupled with one another. Each of the first, second, third, and fourth perfusion chambers 30, 32, 40, 42 includes a plurality of inlets and outlets. For example, each of the perfusion chambers may comprise a first inlet 50 for receiving a stream comprising cells and a first outlet 52 for removing the stream comprising cells. First perfusion chamber 30 has first inlet 50A for receiving cells and first outlet 52A through which cells may be removed. Second perfusion chamber 32 has first inlet 50B and first outlet 52B. Third perfusion chamber 40 has first inlet 50C and first outlet 52C. Fourth perfusion chamber 42 has first inlet 50D and first outlet 52D. In this manner, cells may be introduced into each perfusion chamber (e.g., each of the first, second, third, and fourth perfusion chambers 30, 32, 40, 42) for cell culture and growth and removed, as necessary. Notably, the first inlet 50 and the first outlet 52 may be sealable, which may be reversible, and thus the first inlet 50 and first outlet 52 may be sealed after introduction of cells into a select perfusion chamber.

As will be described further herein, different cells may be introduced into two or more of the perfusion chambers (e.g., each of the first, second, third, and fourth perfusion chambers 30, 32, 40, 42). To this end, the microfluidic device 20 is capable of simulating one or more functions of multiple cell types found in organs in a laboratory setting, where shear stress and environmental conditions may be controlled. Further, each of the perfusion chambers may comprise a second inlet 54 for receiving a fluid medium and a second outlet 56 through which the fluid medium exits.

As shown, first perfusion chamber 30 has second inlet 54A for receiving the fluid medium and second outlet 56A through which fluid medium may exit. Second perfusion chamber 32 has second inlet 54B and second outlet 56B. Third perfusion chamber 40 has second inlet 54C and second outlet 56C. Fourth perfusion chamber 42 has second inlet 54D and second outlet 56D. In this manner, a fluid medium may be introduced into each perfusion chamber (e.g., 30, 32, 40, 42) via second inlets 54A, 54B, 54C, 54D and removed via second outlets 56A, 56B, 56C, and 56D. The fluid medium may flow through each perfusion chamber continuously or intermittently, as will be described further below.

At least one side of each of the perfusion chambers (e.g., a bottom) comprises a gas permeable membrane or layer 60 through which a gas can transfer/pass through and enter into each of the first, second, third, and fourth perfusion chambers (e.g., 30, 32, 40, 42), as will be described further herein. In certain aspects, the gas permeable layer 60 is a porous polymeric layer. For example, the gas permeable layer may be formed of porous polydimethylsiloxane (PDMS). The gas-permeable nature of polydimethylsiloxane (PDMS) allows for fine-tuned control over the oxygen environment at the microscale, making it particularly suitable for use with biological materials such as cultured cells.

The microfluidic device 20 also includes a first micropump, for example, a first microTesla pump 70, disposed with the housing 28 and in fluid communication with the second inlet 54A of the first perfusion chamber 30 and further in fluid communication with the second inlet 54B of the second perfusion chamber 32. Tesla turbines include a stack of concentric disks with predefined gaps and spiral fluid flow and in operation rely on the continuous momentum transfer between the flow and the rotating disks within the fluid boundary layers. In the context of the present disclosure, a microTesla pump has a similar design but on a microscale, where it is configured to process fluids in a microfluidic device. As used herein, “microscale” refers to a structure having at least one dimension that is less than or equal to about 1,000 micrometers (µm), optionally less than or equal to about 900 micrometers, optionally less than or equal to about 800 micrometers (μm), optionally less than or equal to about 700 micrometers (μm), optionally less than or equal to about 600 micrometers (μm), optionally less than or equal to about 500 micrometers (μm), optionally less than or equal to about 400 micrometers (μm), optionally less than or equal to about 300 micrometers (μm), optionally less than or equal to about 200 micrometers (μm), optionally less than or equal to about 150 micrometers (μm), optionally less than or equal to about 100 micrometers (μm), and in certain variations, optionally less than about 50 micrometers. As used herein, reference to a microscale, microfluidic, microchannel, microfluidic channel, or a microstructure, encompasses smaller structures, such as those being on a nanoscale.

In certain variations, the one or more microfluidic channels may have a total volume of less than or equal to about 50 microliters (µL), optionally less than or equal to 40 microliters (µL), optionally less than or equal to 30 microliters (µL), optionally less than or equal to 25 microliters (µL), optionally less than or equal to 20 microliters (µL), optionally less than or equal to 15 microliters (µL), optionally less than or equal to 10 microliters (µL), optionally less than or equal to 9 microliters (µL), optionally less than or equal to 8 microliters (µL), optionally less than or equal to 7 microliters (µL), optionally less than or equal to 6 microliters (µL), optionally less than or equal to 5 microliters (µL), optionally less than or equal to 4 microliters (µL), optionally less than or equal to 3 microliters (µL), optionally less than or equal to 2 microliters (µL), and in certain aspects, optionally less than or equal to 1 microliter (µL).

The first microTesla pump 70 is configured to circulate or pump the fluid medium through the first pair of enclosed perfusion chambers 22, including through the first perfusion chamber 30 and the second perfusion chamber 32. In certain aspects, the first perfusion chamber 30 and the second perfusion chamber 32 are connected to one another (and the first microTesla pump 70) in series, where the fluid medium exits the second outlet 56A of the first perfusion chamber 30 to enter the second inlet 54B of the second perfusion chamber 32.

Further, a second microTesla pump 71 is disposed within the housing 28 and is configured to circulate or pump the fluid medium through the second pair of enclosed perfusion chambers 24, including through the third perfusion chamber 40 and the fourth perfusion chamber 42. The second microTesla pump 71 is thus in fluid communication with the second inlet 54C of the third perfusion chamber 40 and the second inlet 54D of the fourth perfusion chamber 42 and thus is configured to pump or circulate the fluid medium therethrough. It should be appreciated that the fluid medium pumped by the second microTesla pump 71 may be the same fluid medium or a compositionally distinct fluid medium than that circulated and pumped by the first microTesla pump 70. In certain aspects, the third perfusion chamber 40 and the fourth perfusion chamber 42 are connected to one another (and the second microTesla pump) in series, where the fluid medium exits the second outlet 56C of the third perfusion chamber 40 to enter the second inlet 54D of the fourth perfusion chamber 42.

The microfluidic device 20 further includes a microfluidic gas channel 73 in fluid communication with the gas permeable layer 60. As shown, the microfluidic gas channel 73 may be formed or defined in a layer or a substrate 62 below the gas permeable layer 60. This layer 62 may be formed of a polymer, such as polydimethylsiloxane (PDMS). An inlet 72 of the microfluidic gas channel 73 receives a gas. The microfluidic gas channel defines a common manifold region 75 where a first end 74 defines a first branch 76 (corresponding to a region where the first perfusion chamber 30 is above) and a second branch 78 (corresponding to a region where the third perfusion chamber 40 is above). A second end 80 of the common manifold region defines a third branch 82 (corresponding to a region where the second perfusion chamber 32 is above) and a fourth branch 84 (corresponding to a region where the fourth perfusion chamber 42 is above). Each of the first branch 76, second branch 78, third branch 82, and fourth branch 84 may define a serpentine or other flow pattern (e.g., a flow field). Thus, first branch 76 defines a first microfluidic flow field 76A, second branch 78 defines a second microfluidic flow field 78A, third branch 82 defines a second microfluidic flow field 82A, and fourth branch 84 defines a second microfluidic flow field 84A. Each respective microfluidic flow field (76A, 78A, 82A, and 84A) terminates in a respective microfluidic outlet. Thus, the first microfluidic flow field 78A of first branch 76 terminates in first microfluidic outlet 86. The second microfluidic flow field 78A terminates in second microfluidic outlet 88, third microfluidic flow field 82A terminates in third microfluidic outlet 90, and fourth microfluidic flow field 84A terminates in fourth microfluidic outlet 92. Each of the microfluidic outlets 86, 88, 90, and 92 extend into and through the gas permeable layer 60. In this manner, the gas that is introduced into inlet 72 passes through the common manifold

region 75 and into respective branches of the microfluidic channel 73 (namely through first branch 76, second branch 78, third branch 82, and fourth branch 84) to be delivered to the region beneath the perfusion chamber where it can transfer through select regions of the gas permeable layer 60 into each of the first, second, third, and fourth perfusion chambers 30, 32, 40, 42. Notably, while not shown, the microfluidic gas channel 73, including common manifold region 75 and first branch 76, second branch 78, third branch 82, and fourth branch 84 may have valves or other flow regulating components.

In certain variations, the first perfusion chamber 30 receives a first type of cells, while the second perfusion chamber 32 receives a second type of cells distinct from the first type. Likewise, in certain variations, the third perfusion chamber 40 receives the first type of cells (that are also in the first perfusion chamber 30), and the fourth perfusion chamber 42 receives the second type of cells (that are also in the second perfusion chamber 32). In certain variations, the first type of cells and the second type of cells are independently selected from pancreatic beta cells and adipocytes. Pancreatic beta cells may be from cell lines derived from beta cell tumors of the pancreas, such as INS-1, which is a well-known rat-derived insulinoma cell line. For example, 3T3-L1 cells are a suitable line of murine fibroblast-like cells, which under appropriate culture conditions can differentiate into adipocyte-like cells, such as white adipocytes.

In certain aspects, a micropump-driven (e.g., micro-Tesla (µTesla) pump-driven) recirculating microfluidic device is provided that enables investigation into molecular crosstalk between adipocytes and pancreatic beta cells, where the medium flows between adipocytes and pancreatic beta cell components.

In various aspects, during steady-state operations of the microfluidic device, the fluid medium may continuously flow through the first, second, third, and fourth perfusion chambers 30, 32, 40, and 42, pumped by either the first microTesla pump 70 or the second microTesla pump 71. The fluid medium may be a fluid, such as a liquid having dissolved gases therein, which may include natural or synthetic cell culture compositions used to support cell growth. Natural cell culture media may be derived from natural tissue or fluid sources, including blood, plasma, serum, lymph fluids, and the like, while various synthetic cell culture media known in the art may be used, such as minimum essential medium (MEM) or minimum essential medium Eagle, modified Dulbecco’s modified Eagle’s medium (DMEM), fetal bovine serum (FBS), RPMI 1640 medium, and like.

In certain aspects, the first microTesla pump 70 and the first pair of enclosed perfusion chambers 22 define a first microfluidic recirculation loop. In the first recirculation loop, the fluid medium exits the first microTesla pump 70. Thus, the fluid medium is pumped into the second inlet 54A of the first perfusion chamber 30, exits the second outlet 56A of the first perfusion chamber 30, enters the second inlet 54B of the second perfusion chamber 32, exits the second outlet 56B of the second perfusion chamber 32, and then reenters the first microTesla pump 70 for recirculation. Likewise, the second microTesla pump 71 and the second pair of enclosed perfusion chambers 24 may define a second microfluidic recirculation loop, which may be distinct from the first recirculation loop/first microTesla pump 70. In the second recirculation loop, the fluid medium exits the second microTesla pump 71, is pumped into the second inlet 54C of the third perfusion chamber 40, exits the second outlet 56C of the third perfusion chamber 40, enters the second inlet 54D of the fourth perfusion chamber 42, exits the second outlet 54D of the fourth perfusion chamber 42, and reenters the second microTesla pump 71. Notably, the first recirculation loop and the second recirculation loop may be separated and not in fluid communication with one another, and thus, may circulate different fluid medium or have differing conditions (e.g., flow rate).

As will be discussed further herein, in certain variations, the gas that passes through the gas permeable layer 60 comprises oxygen. In this manner, the oxygen level in each of the first, second, third, and fourth perfusion chambers 30, 32, 40, and 42 may be independently controlled. The local environment in each of the perfusion chambers may be selected to be at least one of a hypoxic environment or a normoxic environment. A normoxic environment may be one with a typical concentration of oxygen (e.g., at physiologic conditions, around 2 to 3% by volume of oxygen and in air or atmospheric conditions, around 20 to 21% by volume oxygen), where cells maintain homeostasis. A hypoxic environment is one where the cells are deprived of adequate oxygen levels to maintain homeostasis and thus have lower concentrations of oxygen than normoxic conditions, for example, blood or local medium having an oxygen saturation (SpO₂) level of below about 90%.

Thus, the microfluidic device 20 thus comprises at least two layers, namely the substrate 62 on which the gas microfluidic pathways may be formed, which may be formed of PDMS, and a gas-permeable layer or membrane 60, which may be formed of a porous PDMS. The PDMS substrate 62 may include four chambers/flow fields 76A, 78A, 82A, and 84A associated with each of the first branch 76, second branch 78, third branch 82, and fourth branch 84 that are connected to a main gas chamber or common manifold region 75. Oxygen may be supplied through inlet 72 into the microfluidic gas channel 73 system/chamber, flowing through microchannels into four distinct chambers/flow fields: the first microfluidic flow field 78A of first branch 76 terminates in first microfluidic outlet 86. The second microfluidic flow field 78A of second branch 78 terminates in second microfluidic outlet 88. The third microfluidic flow field 82A of third branch 82 terminates in third microfluidic outlet 90. The fourth microfluidic flow field 84A of fourth branch 84 terminates in fourth microfluidic outlet 92. An upper surface 64 of the substrate 62 (on which the gas microfluidic pathways may be formed) is adjacent to and may be attached to (e.g., bonded) a lower surface 66 of the gas-permeable layer/ membrane 60. A lower surface of 68 of the substrate 62 may be adjacent to and/or attached (e.g., bonded) to an upper surface 94 of a support layer 96, which may be formed of glass, polymer, or metal. In certain aspects, the support layer 96 may be a glass slide.

As shown in FIG. 1A, oxygen may be introduced into the microfluidic device 20 through inlet 72 where it flows into the gas-permeable layer/membrane 60 and then flows into the microfluidic channels 73 of the substrate 62 (e.g., formed of PDMS). An upper surface 67 of the gas-permeable membrane 60 is bonded to the cover component 26, which may be formed of PDMS. In this manner, the solid cover component together with the underlying gas-permeable layer 60 defines each of the first perfusion chamber 30, the second perfusion chamber 32, the third perfusion chamber 40, and the fourth perfusion chamber 42 for cell culture. The housing 28 accommodates the two µTesla pumps 70, 71 embedded within the microfluidic device 20.

In one variation, a first type of cells, such as 3T3L1 cells (which may differentiate into adipocyte-like cells, such as white adipocytes) are injected into first perfusion chamber 30 and into second perfusion chamber 32 vis first inlet 50A and first inlet 50C, respectively, where first outlets 52A and 52C may be pores serving as the outlets for cell injection. Simultaneously, INS1 cells (pancreatic beta cells) may be injected through first inlet 50B of second perfusion chamber 32 and first inlet 50D of fourth perfusion chamber 42, where second outlets 52B and 52D respectively act as outlets for the cell injection media.

Flowing fluid media may be introduced through first pump inlet 102 of the first microTesla pump 70 that has a first pump outlet 104 connected to second inlet 54A of first perfusion chamber 30. Second outlet 56A of first perfusion chamber 30 is connected to second inlet 54B of second perfusion chamber 32. The fluid media can exit from second outlet 56B of second perfusion chamber 32 where it is routed back to first pump inlet 102 of the first microTesla pump 70, thus defining the first recirculation loop for fluid media. In a parallel system within the same microfluidic device 20, fluid medium is injected through second pump inlet 106 into second microTesla pump 71, where it exits the pump via second pump outlet 108. The second pump outlet 108 of second microTesla pump 71 is in fluid communication and connected with second inlet 54C of the third perfusion chamber 40. The second outlet 56D of the third perfusion chamber 40 is connected to second inlet 54D of the fourth perfusion chamber 42. After passing through the fourth perfusion chamber 42, the fluid medium can exit via the second outlet 56D, which may be in fluid communication with or connected to the second pump inlet 106. Thus, the fluid medium is routed back to the second microTesla pump 71, thus defining the second recirculation loop for fluid media.

FIG. 1C shows an example of a microTesla pump 110 that includes a housing 112 into which a rotor component 114 that includes rotors may be inserted. Further, magnets 116 may be inserted into the rotor component 114. After the magnets are inserted into the rotor component 114, the microTesla pump 110 may be disposed within and/or embedded within housing 28 of cover component 26. The microTesla pump 110 may be monitored using a magnetic stirrer.

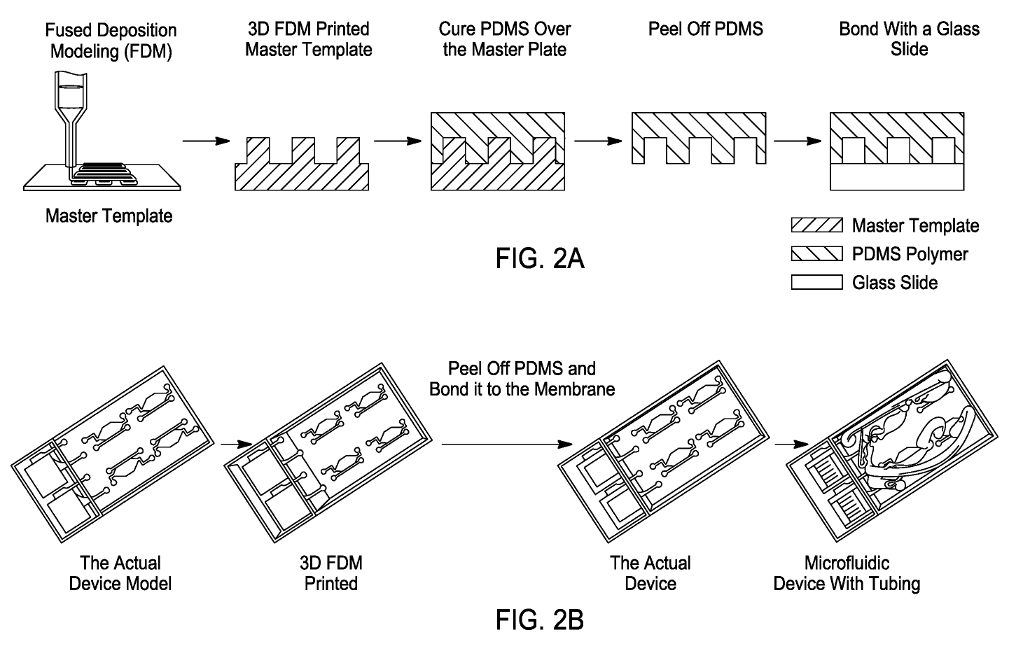

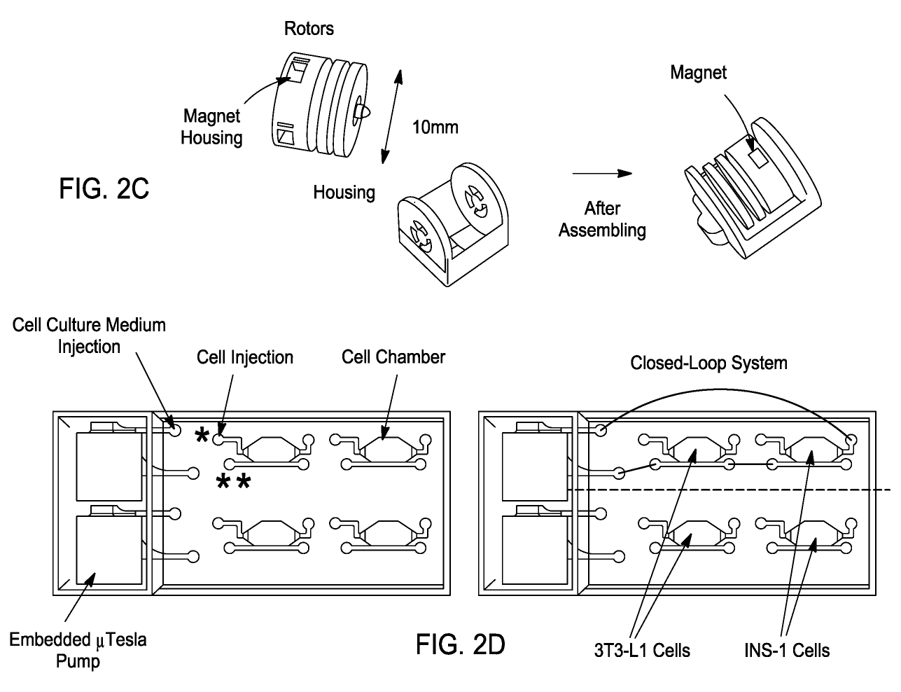

FIGS. 2A–2D show one example of a method for forming a shear force flow (SFF)–enabled microfluidic device according to certain aspects of the present disclosure. FIG. 2A shows a template being fabricated using Fused Deposition Modeling (FDM) additive manufacturing to print a master template. Then, a polymer may applied to a surface of the master template defining the features to be formed. As shown polydimethylsiloxane (PDMS) is disposed over the master template plate by pouring, followed by curing/crosslinking. Once cured, the PDMS layer can be peeled off and then bonded to a planar substrate, such as a glass substrate. FIG. 2B show a microfluidic device design according to certain aspects of the present disclosure having two recirculation loops with four cell perfusion chambers created using Autodesk Inventor software and manufactured using the FDM method. In this design, two µTesla pumps are integrated into the microfluidic device, and the PDMS layer and the pumps were bonded to a glass substrate, as shown on the right with the tubing added.

FIG. 2C shows microTesla pump components and assembly for integration into the microfluidic device, like that shown in FIG. 2B. Each µTesla pump comprises a rotor and housing, which may have a 10 mm size and is printed using a 0.15 mm nozzle following the FDM approach. Two magnets are polarized into the rotor before being assembled into the housing.

FIG. 2D shows images illustrating injection of a cell culture medium into the embedded microTesla pumps connected via silicone tubes to the cell perfusion chambers in the microfluidic device having two recirculation loops with four cell perfusion chambers, enabling media flow over the cultured cells. Cells were introduced into the perfusion chamber through the designated pore (*) and incubated overnight to facilitate attachment. These images illustrate injecting cell culture medium into the embedded µTesla pumps that are connected via silicone tubes to the cell perfusion chambers, enabling media flow over the cultured cells. Cells were introduced into the chamber through the designated inlet/outlet/pores (*) and incubated overnight to facilitate attachment. Subsequently, the µTesla pump was connected to the cell perfusion chamber through a secondary inlet/outlet/pore (**), establishing a continuous media flow over the cells. The symmetrical design of the microfluidic device allows two experiments to be run concurrently with closed-loop perfusion. In certain aspects, such microfluidic devices provide a platform to study the bidirectional crosstalk between adipocytes and beta cells.

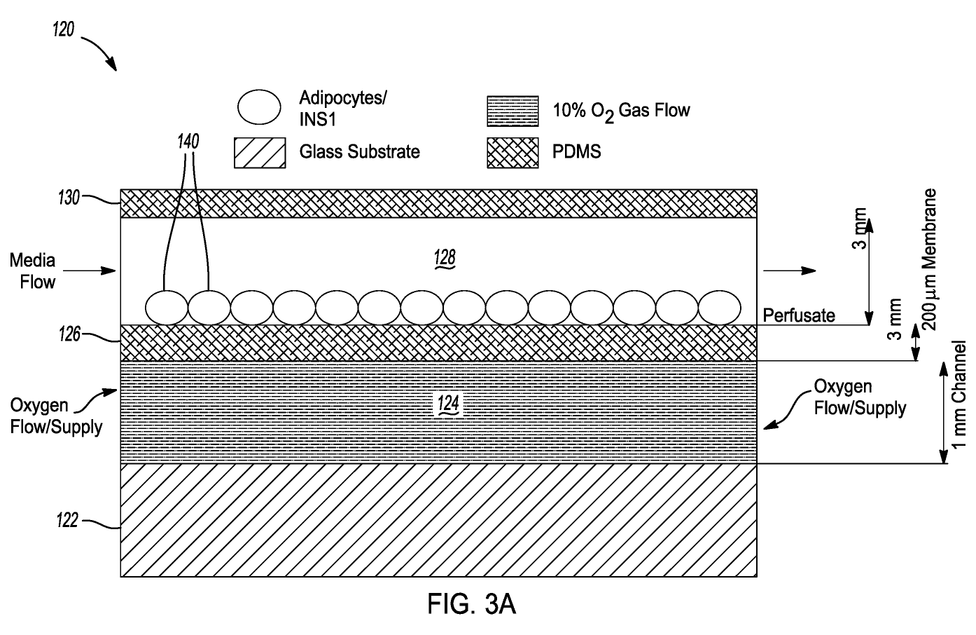

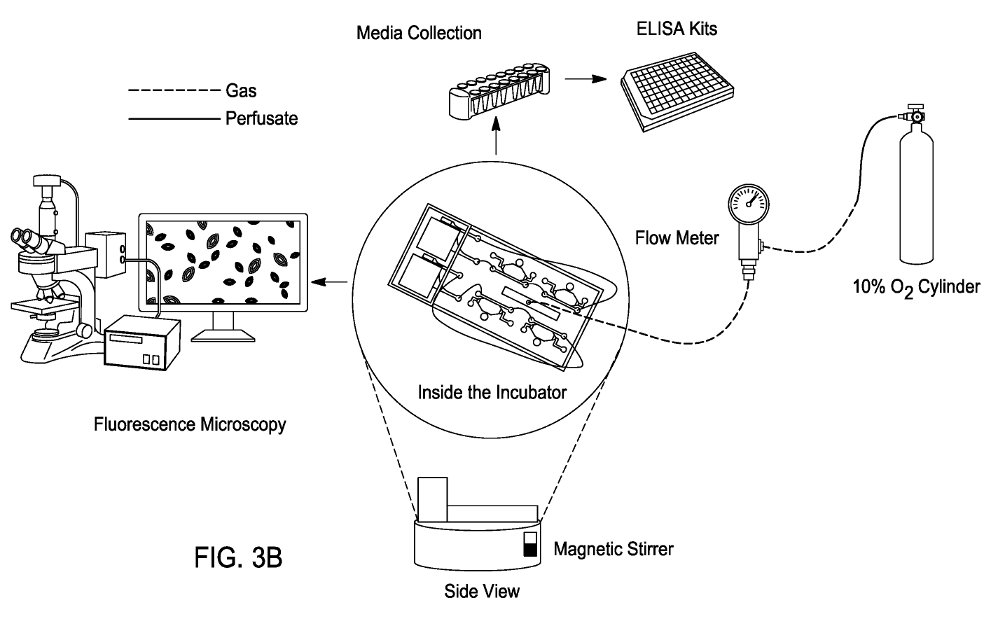

FIGS. 3A–3B show a side sectional view of an example of another perfusion chamber 120 for use in a microfluidic device for regulating flow shear stress on cells. The perfusion chamber 120 includes a support 122, such as a glass slide. Over the support 122, a microfluidic channel 124 or flow path is formed. While not shown, an additional layer may be disposed over the support 122 on which the microfluidic channel 124 is defined. The microfluidic channel 124 is defined between the support 122 and a gas-permeable membrane or layer 126. The microfluidic gas channel 124 may have a height of greater than or equal to about 0.1 millimeters to less than or equal to about 2 millimeters. In one non-limiting variation, the microfluidic channel 124 may have a height of about 1 mm. The gas-permeable membrane/layer 126 may have a thickness of greater than or equal to about 50 micrometers to less than or equal to about 250 micrometers, for example, a thickness of about 200 micrometers. Gas may flow within the microfluidic channel 124 as supplied by tubing or other conduits from a gas source, such as oxygen supplied by a source of oxygen. As shown, a 10% oxygen (O₂) gas may be directed within and through the microfluidic channel 124. A cover layer 130 may be disposed at the top of the perfusion chamber 120 spaced apart from the gas-permeable layer 126. In this manner, an open chamber 128 is defined that has a plurality of cells 140 disposed therein and growing on the gas-permeable layer 126. The open chamber 128 may have a height of greater than or equal to about 2 millimeters to less than or equal to about 4 millimeters, for example, about 3 mm, in one non-limiting variation. A medium may be pumped through the open chamber 128, as described above, such that it enters the open chamber 128 and exits as a perfusate.

In this variation of the microfluidic device incorporating multiple perfusion chambers, like perfusion chamber 120. The perfusion chamber 120 has the glass substrate or support 122 as the first layer, the gas microchannels/microfluidic channel 124 bonded to an upper side of the glass support 122 and to a lower side of the gas-permeable membrane or layer 126. The upper surface of gas-permeable membrane or layer 126 is bonded to the lower surface of the PDMS cover layer 130 forming the gas chambers where cells are cultured.

As shown in FIG. 3B, a schematic of a microscope-based multi-modal platform includes oxygen gas is supplied at a rate of 7.14 sccm from a source of gas (10% oxygen cylinder). The 10% O₂ cylinder may be connected to a flow meter, which is connected to the microfluidic gas channel 124/gas-permeable layer 126/gas chamber 128 of the device. Thus, gas flows through a flow meter to the microfluidic gas channel, which enables proper diffusion through the gas-permeable PDMS substrate/membrane to the cells attached, without detaching the membrane. The plurality of diffusion chambers allow the cells to be fully submerged under flowing media, while the gas-permeable PDMS membrane provides a mechanically stable surface for cell attachment. The tubing of the device is similar to the previous embodiment, where the media is injected through first inlet/port to the µTesla pump and then first outlet port is connected to first inlet of second chamber. First outlet of the second chamber is connected back to the µTesla inlet in a closed-loop manner. The device was then set on a magnetic stirrer to control the rotational speed of the µTesla pump.

The optically transparent PDMS and/or glass substrates allow clear visualization of the cells from the bottom surface of the device, which may measure changes in fluorescence intensity using an inverted microscope. This device may be specifically designed for intensity measurements on the microscope, as capturing images proved challenging due to the PDMS covers, which diminished the light signal from the microscope. Media was collected for ELISA kits and for some experiments, the intensity was measured and analyzed on the microscope using image J.

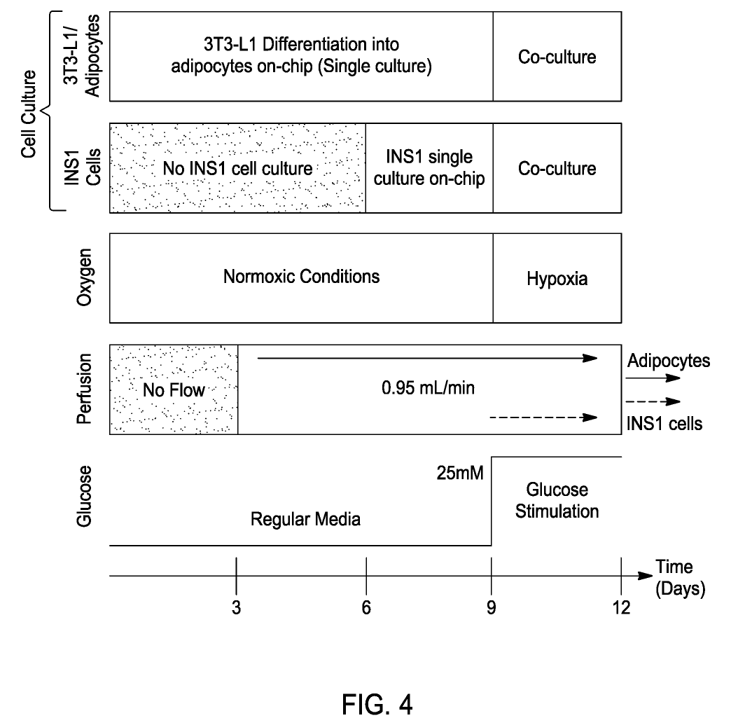

FIG. 4 is a chart illustrating the simultaneous control of oxygen, shear flow, and glucose stimulation at the cells cultured inside such a microfluidic device prepared in accordance with certain aspects of the present disclosure.

The present disclosure also contemplates methods of operating the microfluidic devices, including methods for co-culturing distinct cell types in a microfluidic device under flow shear stress. In one aspect, such a method may include pumping a first fluid medium in a first microfluidic recirculation loop. The first microfluidic recirculation loop may comprise a first pair of enclosed perfusion chambers that includes a first perfusion chamber comprising a first type of cells and a second perfusion chamber comprising a second type of cells. The first microfluidic recirculation loop also comprises a micropump, such as a first microTesla pump to induce flow shear stress conditions on the first type of cells in the first perfusion chamber and the second type of cells in the second perfusion chamber. The method also comprises pumping a second fluid medium in a second microfluidic recirculation loop comprising a second pair of enclosed perfusion chambers. The second pair of enclosed perfusion chambers includes a third perfusion chamber comprising the first type of cells and a fourth perfusion chamber comprising the second type of cells, as well as a second microTesla pump. The pumping induces flow shear stress conditions on the first type of cells in the third perfusion chamber and the second type of cells in the fourth perfusion chamber. The method also includes introducing gas into a microfluidic gas chamber or channel where the gas transfers into each of the first, second, third, and fourth perfusion chambers through a gas permeable membrane, wherein the gas permeable membrane defines at least one wall of the microfluidic channel.

In one aspect, the first fluid medium continuously flows through the first and second perfusion chambers during a first operational mode, such as non-transitory, steady state operations. Likewise, the second fluid medium continuously flows through the third and fourth perfusion chambers during the first operational mode. The pumping of the first fluid, pumping of the second fluid, and the introducing may occur concurrently with one another. In certain aspects, each of the first, second, third, and fourth perfusion chambers comprises a first inlet for receiving a stream comprising either the first type of cells or the second type of cells, a second inlet for receiving either the first fluid medium or the second fluid medium, a first outlet for removing the stream, and a second outlet through which the first fluid medium or the second fluid medium exits.

The stream may thus comprise the first type of cells and the method further comprises passing the stream through the first perfusion chamber and the third perfusion chamber to introduce the first type of cells thereto. After passing the stream through the first perfusion chamber and the third perfusion chamber, the method may include sealing the first outlet of the first perfusion chamber and the first outlet of the third perfusion chamber. In other aspects, the stream comprises the second type of cells and the method further comprises passing the stream through the second perfusion chamber and the fourth perfusion chamber to introduce the second type of cells thereto. After passing the stream through the second perfusion chamber and the fourth perfusion chamber, the method may include sealing the first outlet of the second perfusion chamber and the fourth perfusion chamber. After the sealing, the first, second, third, and fourth perfusion chambers will only have the second inlet and second outlet open for circulating either the first or second fluid medium.

In certain aspects, the first fluid medium passes through the first microfluidic recirculation loop at a flow rate of greater than or equal to about 0.62 mL/minute (where the microTesla pump is operated at 750 rpm) to less than or equal to about 1 mL/minute (where the microTesla pump is operated at 2,000 rpm) and may optionally be about 0.95 mL/minute in certain variations. Likewise, the second fluid medium may pass through the second microfluidic recirculation loop at a flow rate of greater than or equal to about 0.62 mL/minute (where the microTesla pump is operated at 750 rpm) to less than or equal to about 1 mL/minute (where the microTesla pump is operated at 2,000 rpm), for example, optionally at about 0.95 mL per minute. In certain aspects, each microTesla pump may be operated at greater than or equal to about 750 rpm to less than or equal to about 2,000 rpm). For example, two microTesla pumps embedded in a PDMS cover of the microfluidic device can operate at about 1,500 rpm to create a 0.95 mL/min flow and thus shear flow conditions.

In certain aspects, the method may comprise assessing interactions of the first cell type and the second cell type under an influence of shear stress. The assessing interactions of the first cell type and the second cell type under an influence of shear stress may be related to at least two distinct diseases or conditions. For example, the first type of cells and the second type of cells are independently selected from pancreatic beta cells and adipocytes. Thus, a device according to certain aspects of the present disclosure may be designed to regulate flow shear stress (FSS) and oxygen environment for cell culture studies under diabetic and hypoxic conditions. In certain aspects, such microfluidic devices and methods provide a platform to study the bidirectional crosstalk between adipocytes and beta cells.

The gas may comprise oxygen, which is transferred through the gas permeable membrane into each of the first, second, third, and fourth perfusion chambers. In this manner, an oxygen level in each of the first, second, third, and fourth perfusion chambers is independently controlled to be at least one of a hypoxic environment or a normoxic environment.

### Examples

Various embodiments of inventive technology can be further understood by the specific examples contained herein. Specific Examples are provided for illustrative purposes of how to make and use the compositions, devices, and methods according to the present teachings and, unless explicitly stated otherwise, are not intended to be a representation that given embodiments of this invention have, or have not, been made or tested.

The oxygen delivery feature of the microfluidic device may be formed of two polymeric components: the microfluidic channel and the gas-permeable membrane. The photomask is designed using AutoCAD LT 2020 and printed on high-resolution (16,000 dpi) transparency film (FineLine Imaging, Colorado Springs, CO). To create the negative mold master, a 3-inch silicon wafer was thoroughly cleaned and treated with oxygen plasma (Plasmatic Systems, Inc. Plasma-Preen II-862, North Brunswick, NJ). The wafer was then spin-coated (Laurel) with SU-8 2100 photoresist (MicroChem Corporation, MA) to achieve a 200 micrometer (µm) thickness (spun at 1500 rpm for 30 seconds). The wafer was soft-baked at 95° C for 40 minutes, selectively exposed to UV light (315 mJ/cm²), post-exposure baked at 95° C for 14 minutes, and developed.

To fabricate the positive mold of the device, two batches of 10 grams of polydimethylsiloxane (PDMS) (SYLGARD^{TM} 184 kit, Dow Corning) were prepared with a 10:1 ratio of prepolymer to curing agent and degassed under vacuum. The first PDMS batch was spin-coated onto the master to a thickness of 100 µm (spun at 800 rpm for 30 seconds) and cured at 75° C for 15 minutes. The second PDMS batch was then spin-coated on top of the master at the same speed and cured for 2 hours at 75° C, creating a uniform 200 µm thick PDMS layer. Once cured, the PDMS was separated from the master mold and bonded to a 22×40 mm cover glass using oxygen plasma exposure (Plasmatic Systems, Inc. Plasma-Preen II-862, North Brunswick, NJ).

To fabricate the gas-permeable membrane, 10 grams of PDMS was prepared as described previously. The PDMS was then spin-coated onto a new silicon wafer to achieve a thickness of 100 µm, followed by curing at 75° C for 2 hours. Once the PDMS layer was fully cured, a section corresponding to the size of the microfluidic channel was cut from the wafer and transferred onto a transparency film. Using alignment marks, inlet and outlet ports were created in the membrane with a blunted punch tool. After preparing both the gas-permeable membrane and the microfluidic channel, they were exposed to oxygen plasma and bonded together, ensuring that the ports on the membrane aligned with the corresponding inlet and outlet ports on the microfluidic channel substrate.

Pumps were formed as follows. The µTesla pump components were designed using Autodesk Inventor Professional 2023 (AutoDesk. Inc, San Rafael, CA, USA) and printed via fused deposition modeling (FDM) on a custom built Voron Trident 3D printer with polylite ASA filament (Polymaker, Houston, TX, USA). The μTesla pump rotor and the pump housing were printed at 240° C using ASA filament with a 0.15 mm nozzle. The rotor is 1 cm in size, and the housing is designed to hold the rotor and allow it to rotate inside the housing. After printing, two magnets were inserted in the rotor in a polarized fashion. The rotor was inserted into the printed pump housing to assemble the μTesla pump. The fabricated μTesla pump was soaked in 1 M hydrochloric acid (HCL) to increase positive surface charges and then washed with distilled water 5 times. After the HCL incubation, the pump was incubated in 1% sodium dodecyl sulfate (SDS) for 2 days for sterilization and washed with PBS before device encapsulation. The µTesla pump, set to a rotation speed of 1500 rpm, was monitored using a magnetic stirrer to achieve a flow rate of 0.95 mL/min, corresponding to the rate of pancreatic blood flow.

The perfusion chambers are formed. This process is similar to the previously described method. The main inlet, along with outlets, were drilled on opposite sides of chambers, ensuring proper alignment with the oxygen delivery device. To bond the PDMS cover containing the chambers to the gas-permeable membrane, a thin layer of PDMS was applied to the bottom surface of the PDMS cover as an adhesive.

The device is tested for leakage using both water and air. Approximately 3 ml of water is injected into the µTesla pump and into the chambers to verify the proper bonding of the device. For air testing, a cylinder containing 95% O₂/5% CO₂ was connected with a flow rate of 7.14 sccm, matching the flow rate intended for experimental use.

The device is validated as follows. An oxygen probe from Ocean Optics was used to measure the oxygen concentration inside the perfusion chambers. Because the perfusion chambers are symmetrical, measurements were taken from only one chamber. The sensor was calibrated following the manufacturer’s guidelines, using 90% N₂/10% O₂ and 95% O₂/5% CO₂ to represent 10% O₂ and 95% O₂, respectively, as CO₂ can affect the probe's fluorescence. To take measurements, the upper PDMS cover was cut to match the chamber’s size. Oxygen concentration was measured without any flow (static or dynamic) to ensure accuracy. Different oxygen gas mixtures (10% and 95% O₂) were injected through the device, and the probe, mounted on a manual manipulator, was positioned to monitor its location in the x, y, and z planes, with the probe tip precisely touching the center of the chamber.

Cell culture occurs as follows. 3T3L1 cells (ATCC, USA) were cultured in high-glucose DMEM, supplemented with 10% (v/v) bovine calf serum (BCS) and 1% (v/v) penicillin-streptomycin, and incubated at 37° C with 5% CO₂ until they reached confluency. Cells were used up to passage 9. For culturing within the microfluidic device, 1 × 10⁶ 3T3L1 cells were seeded with 1 mL of regular medium and incubated for 3 days until they reached 70% confluency, at which point they were induced to differentiate into adipocytes. Differentiation was initiated by adding media composed of regular 3T3L1 medium, 10 nM Triiodothyronine (T3), 10 µM Troglitazone, 0.25 µM Dexamethasone, and 1 µg/mL Insulin for 3 days, followed by insulin-supplemented media for an additional 3 days. Regular media was reintroduced for another 3 days before experiments commenced. INS1 cells were added on the 9th day of differentiation and cultured for 3 days until they reached 90% confluency before co-culturing with the adipocytes. Cell viability was assessed using the Live/Dead viability kit (ThermoFisher Scientific, USA) and analyzed using ImageJ. All measurements were performed in triplicate, and cell viability was calculated as previously described.

Mitochondrial ATP is quantified as follows. ATP LW and red live cell fluorescence dye (BioTracker) were utilized to measure the ATP levels in the mitochondria of adipocytes and INS1 cells. Samples from both cell lines were stained with 5 µM BioTracker dye and incubated for 30 minutes at 37° C and 5% CO₂ prior to imaging. The samples were incubated for a total of 24 hours, with images captured at 0, 3, 12, and 24 hours using an Olympus IX75 microscope with 10x magnification. Fluorescence signal intensity was analyzed and quantified using ImageJ software, with values normalized to the 0-hour time point.

Reactive oxygen species quantification is conducted as follows. Reactive oxygen species (ROS) were qualitatively assessed using the cell permeant 2’,7’-dichlorodihydrofluorescein diacetate (H₂DCFDA) from ThermoFisher Scientific. Following the manufacturer’s instructions, DCFDA was dissolved in DMSO to obtain a 10 mM standard solution, which was then diluted 2 times to achieve a final concentration of 10 µM. 1.2 µl of 10 µM solution was added to the regular media and incubated for 1 h. The samples were incubated for 24 hours, with images taken at 0, 3, 12, and 24 hours using an Olympus IX75 microscope with 10x magnification. Fluorescence signal intensity was analyzed and quantified using ImageJ software and normalized to the 0-hour time point.

IL-6 concentrations were measured using the IL-6 Mouse ELISA Kit (Invitrogen, USA) following the manufacturer’s instructions. Samples were collected 24 hours after adding 50, 100, 300, 1000, and 10000 pg/mL of insulin to the flowing media. Absorbance was measured using a BioTek Eon Microplate reader at 450 nm. TNF-α, insulin, and adiponectin secretion were quantified using the protocol described by TNF alpha mouse, insulin rat, and mouse adiponectin ELISA kits (Invitrogen, USA), with absorbance measured at 450 nm. The secretion of inflammatory cytokines per cell was quantified by dividing the concentration by the total number of cells in each chamber (35000 cells/chamber).

Calcium Influx Detection occurs as follows. For calcium responses, cells were stained with 5 µM Fura-2 AM (ThermoFisher, USA) for 30 mins following overnight incubation with insulin concentrations of 50, 100, 300, 1000, and 10000 pg/mL insulin, and glucose concentrations of 5.5 mM, 12.5 mM, and 25 mM. Subsequently, the medium was replaced with glucose Krebs-Ringer buffer. The devices were then placed under fluorescence microscopy and incubated for 5 minutes before stimulating with 12.5 mM glucose for 24 hrs. FURA fluorescence intensities were measured at 340 nm and 380 nm wavelengths after 24 hrs., and the 340/380 nm intensity ratio was calculated to represent intracellular calcium levels.

Statistical analysis for all experiments were conducted at least in triplicates and repeated three times. The data represent the mean ± S.E.M. of three independent experiments. Statistical analysis was carried out using one-way ANOVA and regression analysis. The differences between the two sets of data were considered significant at p-value < 0.05.

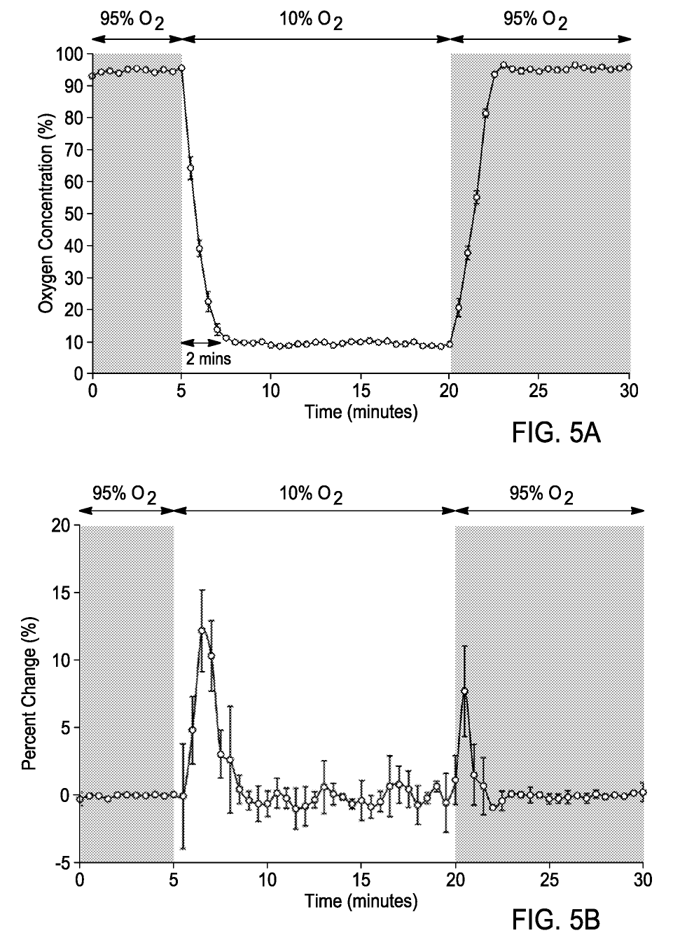

Referring to FIG. 5A, oxygen concentration readings were taken using two different supply concentrations. The microfluidic device prepared as described above demonstrated that after applying a 95% O₂ supply, it took approximately 2 minutes to reach equilibrium when switching to a 10% O₂ supply, with levels ranging between 8.9% and 11.2%. When the 95% O₂ supply was reintroduced, it took about 3 minutes to stabilize again. As shown in FIG. 5B, the percent change was around 0% during the initial 95% O₂ supply, increasing to approximately 12% after switching to 10% O₂. It then fluctuated between –1.2% and 1.6% until the 95% O₂ was reapplied, causing the percent change to rise to 8.9% before returning to equilibrium around 0%.

To gain deeper insights into the effects of hypoxia on the interaction between inflammatory cytokine secretion from adipocytes and insulin and calcium release from Beta INS1 cells, both cell types were co-cultured on a microfluidic chip. Under normoxic conditions, the cells were cultured on a glass substrate pre-coated overnight with collagen. For hypoxic conditions, the cells were cultured on a 200 micrometer (µm) PDMS membrane also coated with collagen. Media was introduced into the µTesla pump and circulated for approximately 5 minutes to eliminate air bubbles before being connected to the cell culture perfusion chambers in the microfluidic device. A closed-loop system was used to recirculate the same media over both chambers (containing adipocytes and INS1 cells), with media being refreshed every 8 hrs. A 10% O₂ cylinder was connected to the device through the gas chamber, and air flowed through the microchannels between the 200 µm PDMS membrane and the glass substrate. Oxygen diffusion occurred between the air through the porous PDMS to the cells and media.

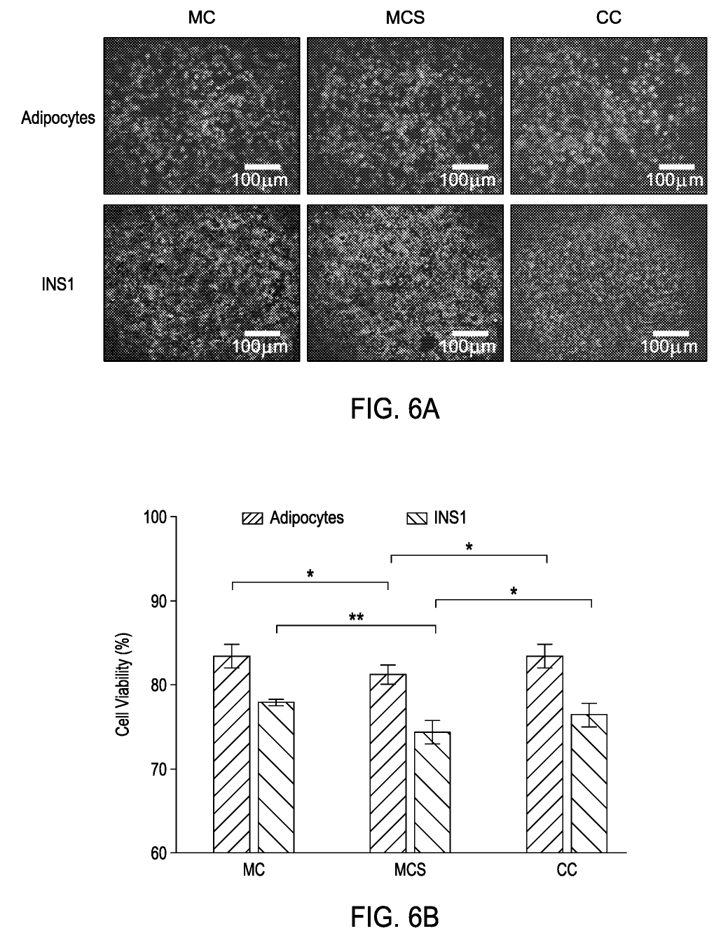

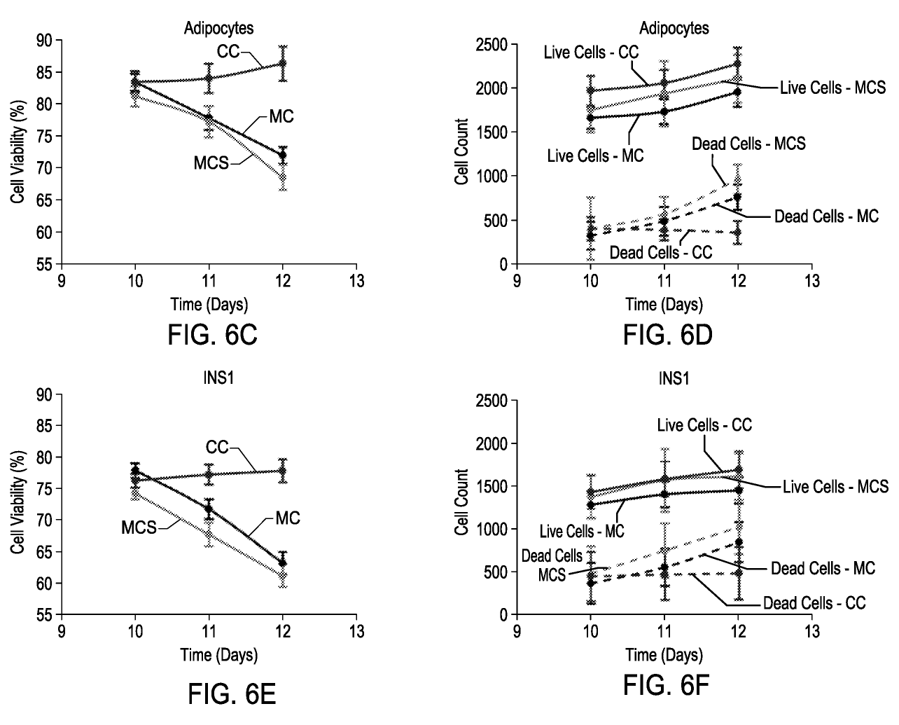

Normoxic studies are conducted as follows. The overall viability of differentiated adipocyte and INS1 cells under specific MC, MCS, and CC, microfluidic culture conditions was quantified using a Live/Dead Viability Kit (ThermoFisher Scientific, USA) (FIG. 6A). Cell counts were quantified using Image J, and then cell viability was calculated from the microfluidic cultures. FIG. 6B illustrates a minor reduction in cell viability for adipocytes and INS1 monocultures under shear, from 83.4% and 78.0% to 81.2% and 74.3%, respectively (p-value<0.05). In contrast, the co-culture experiments (CC with perfusion and shear) showed higher cell viabilities restoring adipocytes and INS1 cells back to 83.4% and 76.3%, respectively (p-value < 0.05).

For a more nuanced view of the viability data, the live and dead cells are followed separately over the course of 3 days. Overall viability was lower for monocultured cells under shear (MCS) compared to no shear (MC), as expected (FIGS. 6C and 6E). However, co-cultured cells maintained and even increased viability over the same duration. When individual life and death counts were examined, both live and dead cell counts increased over time in monocultured cells (FIGS. 6D and 6F). For co-cultured adipocytes and INS1, however, live cell counts increased while the number of dead cells remained relatively stable (FIGS. 6D and 6F). After 12 days of on-chip culturing, the dead cell count for adipocytes and INS1 co-culture remained constant while monocultures were roughly doubled. These results suggest that cell-cell interaction facilitated by the co-cultured perfusion helped to halt cell death in both adipocytes and INS1 cells, providing more viable cells for the following cytokine experiments.

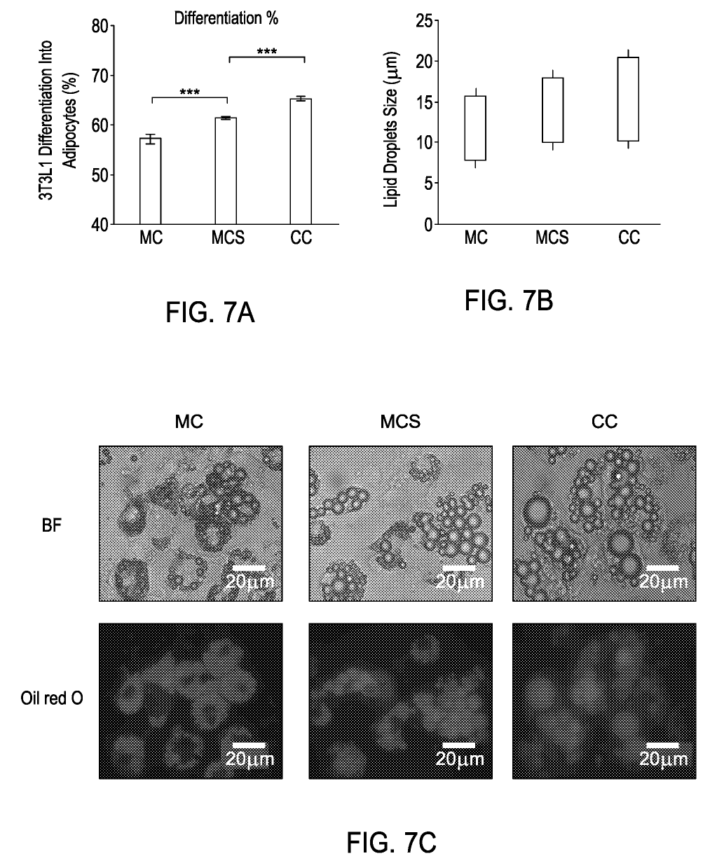

Prompted by the improved viability under co-culturing, whether shear stress had any effect on the differentiation of 3T3L1 into adipocytes under the same conditions is investigated. Results indicate that shear stress and co-culturing significantly improve monoculture differentiation of 3T3L1 cells, with a higher percentage of lipid-containing cells observed under shear conditions (MCS) compared to no shear conditions (MC) (60.4% versus 55.5%) (p-value < 0.001) (FIGS. 7A and 7C). Furthermore, co-culturing with INS1 cells (CC) further increased in 3T3L1 differentiation from 60.4% to 64.0%, (p-value < 0.001) (FIG. 7A). Additionally, shear stress is associated with increased lipid droplet sizes, as the average lipid droplets measured 11.8 µm without shear (MC) versus 14 µm under shear (MCS) and 15.5 µm under co-culture (CC) (FIGS. 7B and 7C).

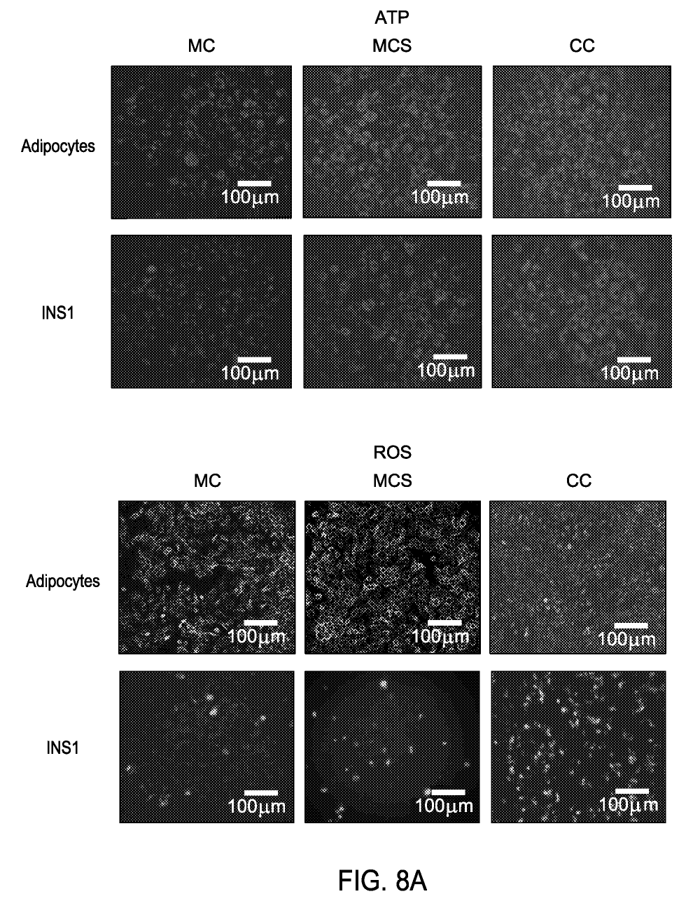

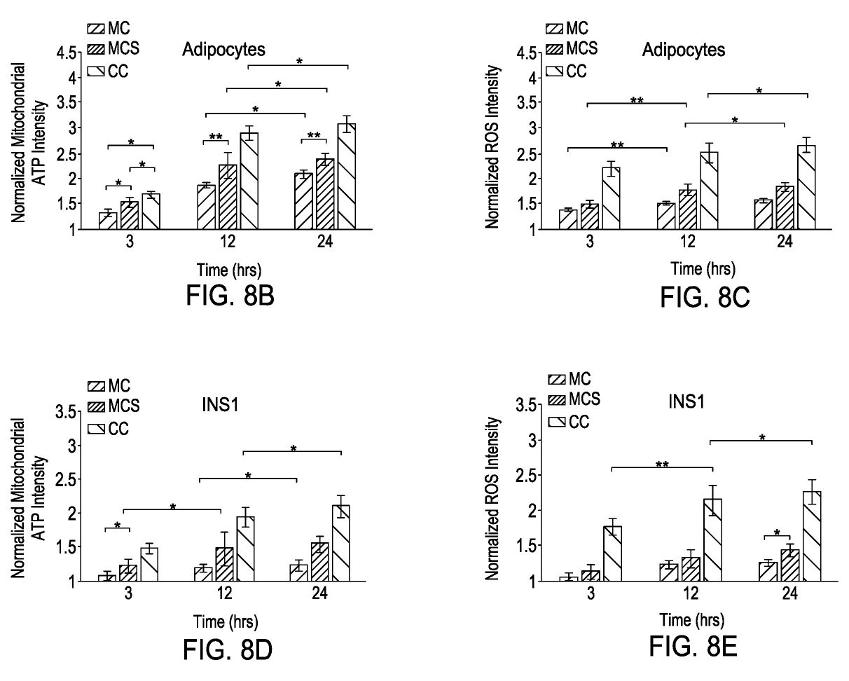

The improved differentiation suggested an investigation that was conducted into adipocyte and INS1 metabolism under shear and co-culturing conditions via mitochondrial ATP levels and reactive oxygen species (ROS) (FIGS. 8A–8E). Differentiated adipocytes exhibited higher mitochondrial ATP levels compared to 3T3L1 cells, both with and without shear stress, attributable to the metabolic activity of mature versus MCS at 12 hrs.), as did co-culture conditions (CC) (p<0.001, MCS versus CC at 12 hrs.), (FIGS. 8A and 8B). Furthermore, the increases occurred primarily in the first 12 hours, as levels at 24 hours were only moderately higher. A similar trend was observed for ROS production in adipocytes (FIGS. 8A and 8C), with the co-culture (CC) showing more prominent increases than that with shear stress alone (p<0.001, MCS versus CC at 12 hrs.). This suggests that shear stress and co-culturing, to a higher degree, trigger changes in metabolic pathways, leading to enhanced respiration and energy production evidently at the mitochondrial level.

On the other hand, the metabolic activities of INS1 cells were also monitored. Shear stress increased INS1 ATP levels significantly (p<0.05, MC versus MCS at 12 hrs.), as did co-culture conditions (CC) to a greater degree (p<0.001, MCS versus CC at 12 hrs.), (FIGS. 8A and 8D). Similar to adipocytes, the increases occur primarily in the first 12 hours. The trend for ROS followed the ATP rises, (FIGS. 8A and 8E). And both INS1 ATP and ROS benefited from co-culturing (CC) more than shear stress alone (p<0.001, MCS versus CC at 12 hrs.). Thus, factors beyond shear from perfusion are elevating both INS1 and adipocytes to a heightened metabolic state, perhaps as a result of their increased inflammatory and insulin secretions.

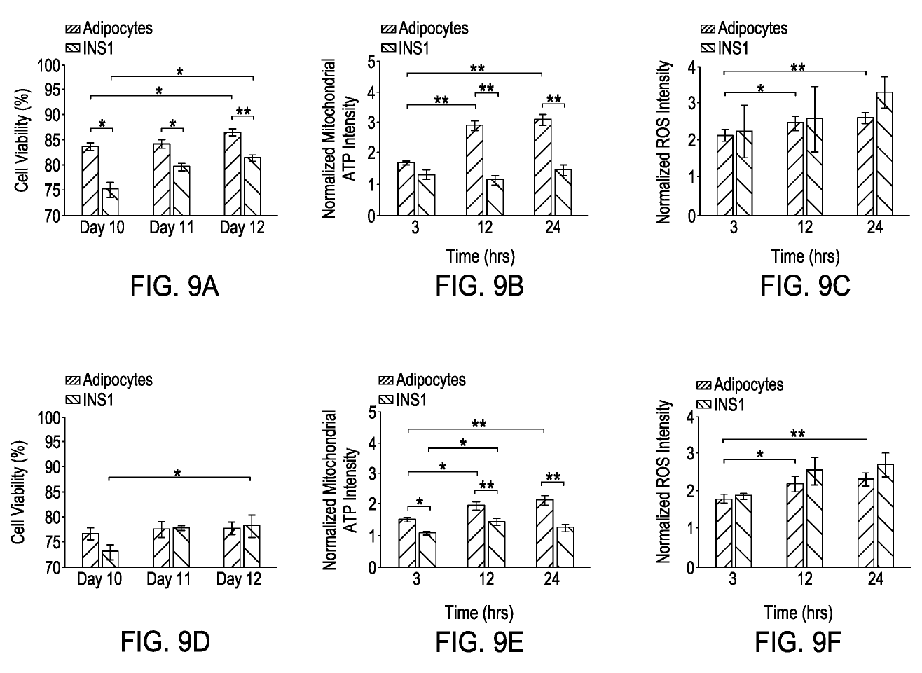

Normoxic and hypoxic studies are conducted. Referring to FIGS. 9A–9F, the viability and metabolism of adipocytes and INS1 cells were evaluated under both normoxic and hypoxic co-culture conditions. For adipocytes, cell viability was monitored over three days following the differentiation of 3T3L1 cells (FIG. 9A). Under hypoxic conditions, viability was significantly lower, reaching values of 75.03 ± 1.46%, 79.54 ± 0.69%, and 81.24 ± 0.68% (p-value < 0.05) on Days 10, 11, and 12, respectively (FIG. 9A). In contrast, normoxic conditions yielded higher viability rates of 83.53 ± 0.72%, 84.04 ± 0.87%, and 86.33 ± 0.65% (p-value < 0.05) over the same period (FIG. 9A). INS1 cells showed a similar pattern on Day 10, with viability at 76.56 ± 1.19% under normoxia and 72.97 ± 1.48% under hypoxia (FIG. 9D). However, on Days 11 and 12, the viability rates were nearly identical for both conditions, with normoxia showing 77.39 ± 1.61% and 77.58 ± 1.26%, and hypoxia showing 77.65 ± 0.48% and 78.06 ± 2.21%, respectively (FIG. 9D).

The metabolic activity of adipocytes and INS1 cells was assessed by measuring mitochondrial ATP levels and reactive oxygen species (ROS) intensity in both cell lines. The trend in mitochondrial ATP levels was consistent between adipocytes and INS1 cells, with higher ATP values observed under normoxic conditions compared to hypoxia (FIGS. 9B and 9E). After 24 hours, the normalized mitochondrial ATP intensity for adipocytes was 3.06 ± 0.16 in normoxia and 1.44 ± 0.17 in hypoxia (p-value < 0.001) (FIG. 9B). For INS1 cells, the values were 2.09 ± 0.14 in normoxia and 1.21 ± 0.11 in hypoxia (p-value < 0.001) (FIG. 9E). Conversely, ROS intensity exhibited an opposite pattern, with higher values under hypoxia than normoxia (FIGS. 9C and 9F). In adipocytes, the normalized ROS intensity was 2.66 ± 0.15 in normoxia and 3.36 ± 0.42 in hypoxia after 24 hours (FIG. 9C). For INS1 cells, the ROS intensity was 2.3 ± 0.17 in normoxia and 2.69 ± 0.32 in hypoxia over the same period (FIG. 9F).

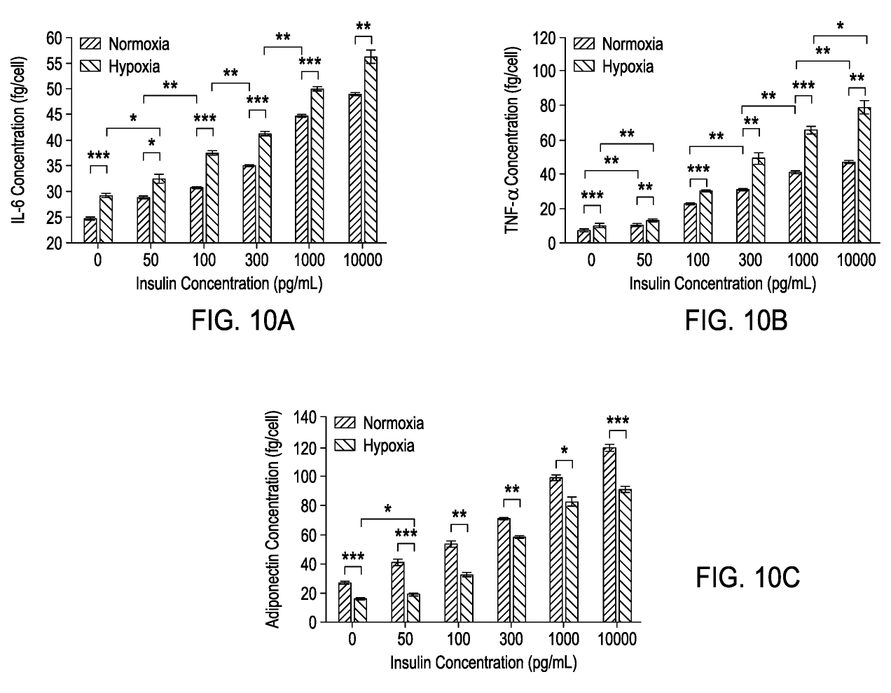

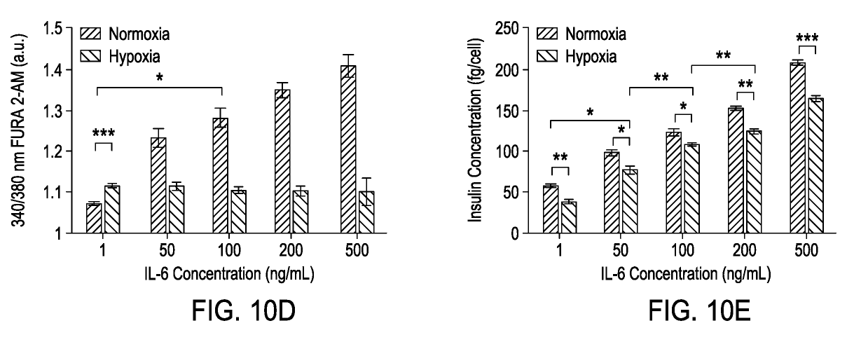

To investigate the effect of insulin on inflammatory cytokine secretion from adipocytes, cells were exposed to varying insulin concentrations (0, 50, 100, 300, 1000, and 10000 pg/mL), with media collected after 24 hrs. (FIGS. 10A, 10B and 10C). Quantification of IL-6, TNF-α, and adiponectin was performed using ELISA kits. Results indicated that under hypoxic conditions, IL-6 secretion increased significantly, with values reaching 29.21 ± 0.38 fg/cell at 0 pg/mL insulin, 41.36 ± 0.38 fg/cell at 300 pg/mL, and 56.23 ± 1.32 fg/cell at 10,000 pg/mL (FIG. 10A). Under normoxia, IL-6 secretion was lower, measuring 24.75 ± 0.33 fg/cell at 0 pg/mL insulin (p-value < 0.001), 35.04 ± 0.14 fg/cell at 300 pg/mL (p-value < 0.001), and 48.93 ± 0.29 fg/cell at 10,000 pg/mL (p-value < 0.01) (FIG. 10A). A similar trend was observed for TNF-α, with levels reaching 77.71 ± 3.73 fg/cell under hypoxia and 46.39 ± 0.98 fg/cell under normoxia at 10,000 pg/mL insulin (p-value < 0.01) (FIG. 10B). In contrast, adiponectin secretion was reduced under hypoxic conditions, with values at 15.8 ± 0.29 fg/cell, 58.49 ± 0.85 fg/cell, and 91.29 ± 1.84 fg/cell at 0, 300, and 10,000 pg/mL insulin, respectively, compared to normoxia, where concentrations were higher at 26.94 ± 0.97 fg/cell (p-value < 0.001), 71.05 ± 1.5 fg/cell (p-value < 0.01), and 119.73 ± 2.42 fg/cell (p-value < 0.001) (FIG. 10C). These findings suggest that insulin levels modulate inflammatory cytokine secretion from adipocytes under hypoxic conditions, potentially contributing to metabolic dysregulation. The greatest impact of insulin modulation was on TNF-α reaching higher concentrations compared to IL-6 and adiponectin under hypoxia.

IL-6 has been shown to directly influence calcium and insulin secretion from INS1 cells. To explore this crosstalk, INS1 cells were stimulated with IL-6 concentrations of 1, 50, 100, 200, and 500 ng/mL (FIGS. 10D and 10E). Under hypoxic conditions, calcium secretion remained relatively stable across these concentrations, with a maximum value of 1.09 ± 0.03 a.u. at 500 ng/mL IL-6 (FIG. 10D). However, under normoxic conditions, a positive correlation between IL-6 concentration and calcium secretion was observed, with values increasing from 1.17 ± 0.007 a.u. at 1 ng/mL to 1.41 ± 0.027 a.u. at 500 ng/mL (FIG. 10D). Insulin secretion followed a similar trend under normoxia, reaching 207.86 ± 3.04 fg/cell at 500 ng/mL IL-6 (FIG. 10E). In contrast, under hypoxia, insulin secretion displayed a distinct pattern, with concentrations rising from 37.17 ± 2.17 fg/cell at 1 ng/mL (p-value < 0.01) to 164.13 ± 4.03 fg/cell at 500 ng/mL (p-value < 0.001) (FIG. 10E). These findings highlight a direct regulatory effect of IL-6 from adipocytes on INS1 cells, suggesting a complex circulatory network between the inflammatory cytokine secretion from adipocytes and the modulation of calcium and insulin secretion in INS1 cells. To be more specific, INS1 cells insulin was modulated by IL-6 but with concentrations for hypoxia lower than that of normoxia.

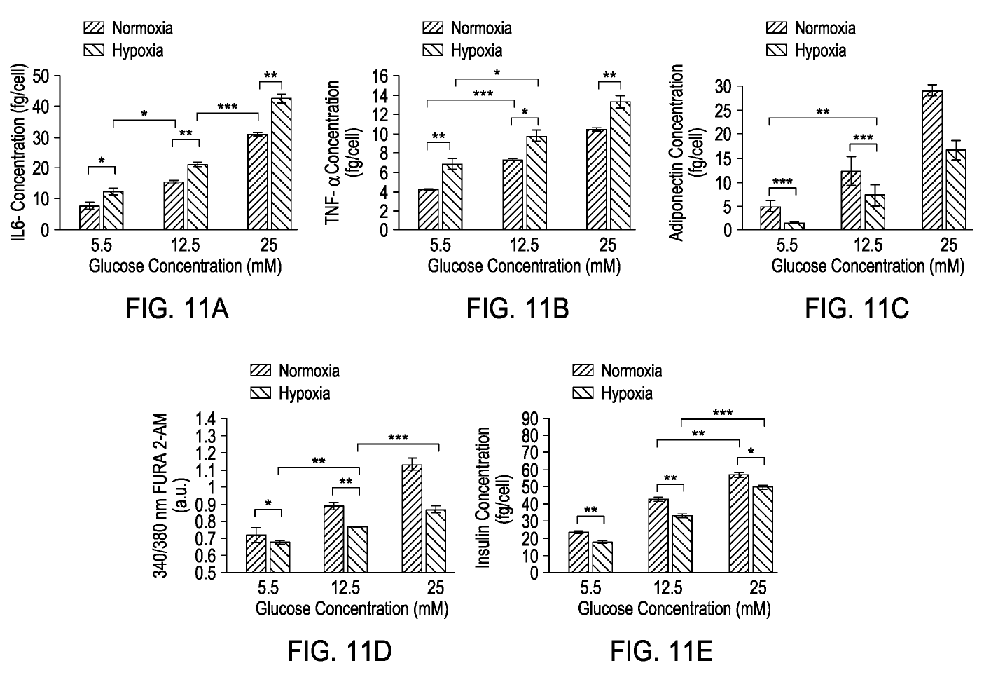

To investigate the interaction between inflammatory cytokine secretion from adipocytes and the calcium and insulin from INS1 cells in a diabetic individual under hypoxic conditions (10% O₂), a glucose modulation study was conducted using concentrations of 5.5, 12.5, and 25 mM (FIGS. 11A–11E). Both cell lines were co-cultured on a microfluidic chip as described above and subjected to shear stress. The results revealed that IL-6 secretion increased exponentially under both normoxic and hypoxic conditions (FIG. 11A). Specifically, IL-6 levels rose from 7.59 ± 0.37 fg/cell, 15.61 ± 0.32 fg/cell, and 30.94 ± 0.42 fg/cell in normoxia to 12.42 ± 1.06 fg/cell (p-value < 0.05), 21.15 ± 0.63 fg/cell (p-value < 0.01), and 42.55 ± 1.53 fg/cell (p-value < 0.01) in hypoxia for glucose concentrations of 5.5 mM, 12.5 mM, and 25 mM, respectively (FIG. 11A). A similar pattern was observed for TNF-α, though at lower concentrations, with levels reaching 10.42 ± 0.26 fg/cell in normoxia and 13.3 ± 0.66 in hypoxia at 25 mM glucose (p-value < 0.05) (FIG. 11B). In contrast, adiponectin secretion demonstrated an inverse relationship, with higher concentrations under normoxic conditions compared to hypoxia, and an exponential increase across the glucose concentrations (FIG. 11C). Adiponectin levels decreased from 4.43 ± 1.19 fg/cell, 11.33 ± 2.7 fg/cell, and 26.94 ± 0.97 fg/cell in normoxia to 1.43 ± 0.09 fg/cell (p-value < 0.001), 6.63 ± 2.05 fg/cell (p-value < 0.001), 15.49 ± 1.85 fg/cell in hypoxia for glucose concentrations of 5.5, 12.5, and 25 mM, respectively (FIG. 11C).

For INS1 cells, both calcium (FURA) intensity and insulin secretion exhibited a linear increase under normoxic and hypoxic conditions, though the slope was steeper under hypoxia (FIG. 11D). Specifically, calcium intensity rose from 0.72 ± 0.05 a.u. at 5.5 mM glucose to 1.14 ± 0.04 a.u. at 25 mM glucose under normoxia, which was higher than the increase observed under hypoxia, where it ranged from 0.68 ± 0.01 a.u. at 5.5 mM glucose (p-value < 0.05) to 0.87 ± 0.02 at 25 mM glucose (FIG. 11D). Similarly, insulin concentrations were measured using an ELISA kit, showing an increase from 23.41 ± 0.67 fg/cell at 5.5 mM glucose to 57.18 ± 1.34 fg/cell at 25 mM glucose under normoxia, and from 17.66 ± 0.67 fg/cell at 5.5 mM glucose (p-value < 0.01) to 49.94 ± 1.45 fg/cell at 25 mM glucose (p-value < 0.05) under hypoxia (FIG. 11E). These findings suggest a complex interplay where insulin secretion from INS1 cells under hypoxic conditions promotes IL-6 and TNF-α secretion from adipocytes while inhibiting adiponectin secretion.

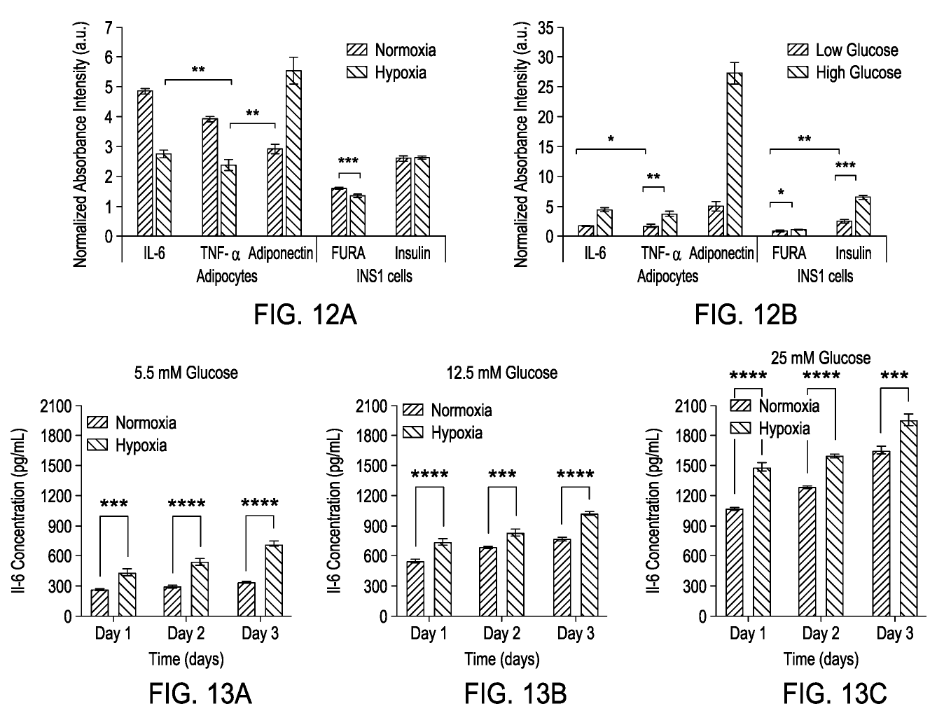

Hypoxia and glucose were key factors influencing the behavior of adipocytes and INS1 cells in the microfluidic co-culture model described herein, particularly for diabetic conditions. To better understand the effects of hypoxia on these co-cultured cells, the absorbance intensity of inflammatory cytokines from adipocytes and calcium and insulin from INS1 cells are normalized, under high glucose (25 mM) to low glucose (5.5 mM) and compared to those under normoxia after an extended culture with glucose (FIG. 12A). The normalized intensities serve as the impact ratio. The findings show that hypoxic conditions led to a reduction in IL-6 and TNF-α secretion from adipocytes, while simultaneously promoting adiponectin secretion (FIG. 12A). Specifically, the normalized absorbance intensity for IL-6 dropped from 4.85 ± 0.1 a.u. to 2.75 ± 0.14 a.u., and for TNF-α, it decreased from 3.91 ± 0.08 a.u. to 2.37 ± 0.19 a.u. Conversely, adiponectin secretion increased significantly from 2.91 ± 0.16 a.u. to 5.54 ± 0.44 a.u. For INS1 cells, the calcium level (FURA) slightly decreased under hypoxia from 1.61 ± 0.03 a.u. to 1.35 ± 0.05 a.u., while insulin levels remained stable between normoxia and hypoxia around 2.62 ± 0.05 a.u (FIG. 12A).

The impact of hypoxia on diabetic individuals was further evaluated by comparing co-cultured cells with low and high glucose levels both under hypoxic conditions (FIG. 12B). The findings indicate that glucose significantly influenced adiponectin secretion from adipocytes while also enhancing IL-6 and TNF-α secretion (FIG. 12B). For INS1 cells, the presence of glucose under hypoxia more effectively stimulated insulin secretion compared to conditions with low glucose. This suggests that at high glucose concentrations, adiponectin promotes insulin secretion, offering protective effects against inflammation in a hypoxic environment.

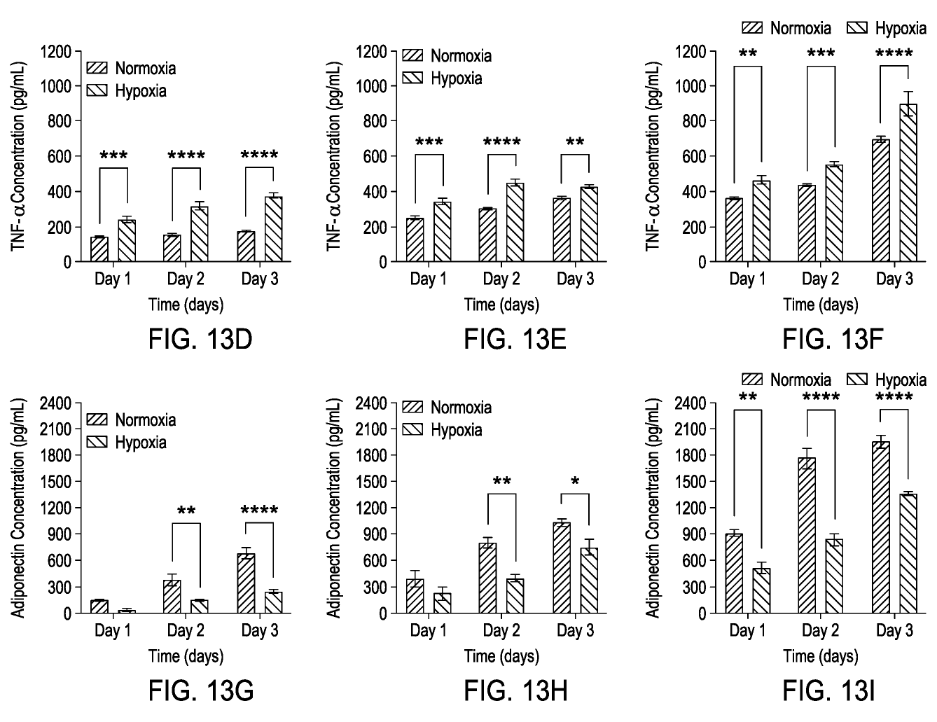

In further experiments conducted in the microfluidic co-culture model described above, further investigation of the impact of insulin secretion from beta cells on adipokine secretion from adipocytes under diabetic and hypoxic conditions is conducted. Adipocytes and beta INS1 cells are co-cultured for 3 days and subjected to glucose modulations under hypoxic conditions. FIGS. 13A–13I show IL-6, TNF-α, and adiponectin secretion from co-cultured adipocytes when exposed to glucose modulations (5.5 mM glucose, 12.5 mM glucose, or 25 mM of glucose) under normoxic and hypoxic conditions. More specifically, FIGS. 13A–13C show adipocyte IL-6 response to glucose incubation in normoxic and hypoxic conditions for 3 days. Adipocytes were incubated with 5.5 mM glucose (FIG. 13A), 12.5 mM glucose (FIG. 13B), or 25 mM of glucose (FIG. 13C) for 24 hours under normoxic and hypoxic conditions and IL-6 levels in the media were measured. n=3 independent experiments. FIGS. 13D–13F show adipocyte TNF-α response to glucose incubation in normoxic and hypoxic conditions for 3 days. Adipocytes were incubated with 5.5 mM glucose (FIG. 13D), 12.5 mM glucose (FIG. 13E), or 25 mM of glucose (FIG. 13F) for 24 hours under normoxic and hypoxic conditions and TNF-α levels in the media were measured. n=3 independent experiments. Finally, FIGS. 13G–13I show adipocyte adiponectin levels in response to glucose incubation in normoxic and hypoxic conditions for 3 days. Adipocytes were incubated with 5.5 mM glucose (FIG. 13G), 12.5 mM glucose (FIG. 13H), or 25 mM of glucose (FIG. 13I) for 24 hours under normoxic and hypoxic conditions and adiponectin levels were measured. n=3 independent experiments. Two-way ANOVA with Tukey multiple comparison tests was used. Data are presented as mean ± SEM. *P<0.05, **P<0.01, ***P<0.001, ****P<0.0001. n stands for different experiments conducted in separate days. As shown in FIGS. 13A–13I, co-cultured adipocytes respond to glucose modulations under hypoxia.

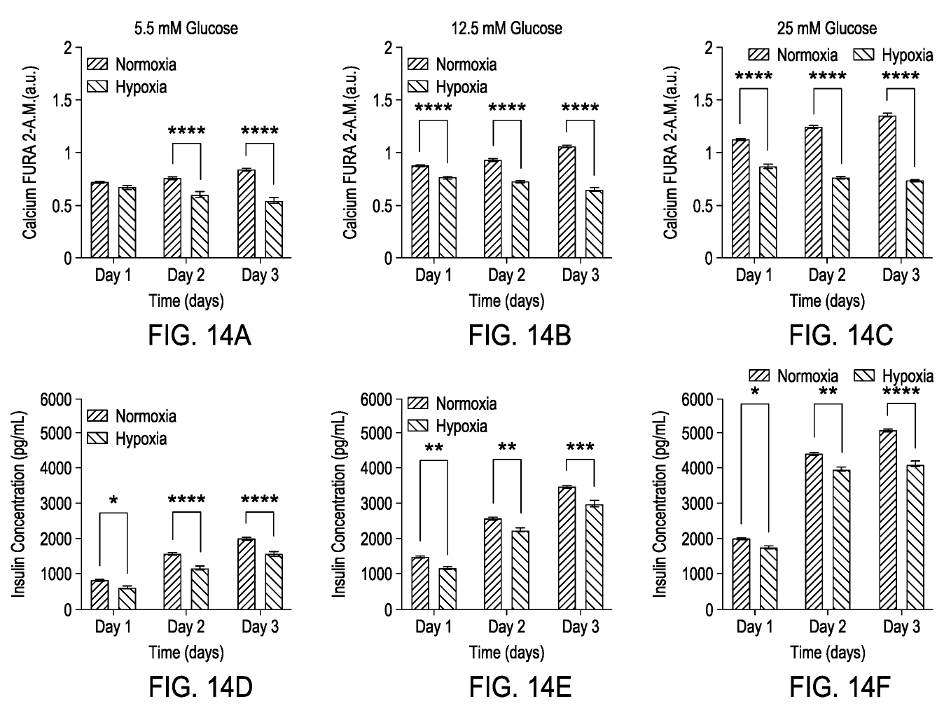

FIGS. 14A–14F show calcium influx detection (by FURA 2) and insulin concentration from co-cultured beta INS1 cells when exposed to glucose modulations (e.g., 5.5 mM glucose, 12.5 mM glucose, or 25 mM of glucose) under normoxia and hypoxia conditions over 3 days. More specifically, FIGS. 14A–14C show beta INS1 calcium response to glucose incubation in normoxic and hypoxic conditions for 3 days. Beta INS1 cells were incubated with 5.5 mM glucose (FIG. 14A), 12.5 mM glucose (FIG. 14B), or 25 mM of glucose (FIG. 14C) for 24 hours under normoxic and hypoxic conditions and calcium levels in the media were measured. n=3 independent experiments. FIGS. 14D–14F show beta INS1 insulin response to glucose incubation in normoxic and hypoxic conditions for 3 days. Beta INS1 cells were incubated were incubated with 5.5 mM glucose (FIG. 14D), 12.5 mM glucose (FIG. 14E), or 25 mM of glucose (FIG. 14F) for 24 hours under normoxic and hypoxic conditions and insulin levels in the media were measured. n=3 independent experiments. Two-way ANOVA with Tukey multiple comparison tests was used. Data are presented as mean ± SEM. *P<0.05, **P<0.01, ***P<0.001, ****P<0.0001. n stands for different experiments conducted in separate days.

Co-culturing adipocytes and beta INS1 cells for 3 days provided deeper insights into the impact of insulin secretion from beta cells on adipokine secretion from adipocytes under diabetic and hypoxic conditions. Over the first 2 days with 5.5 mM and 12.5 mM glucose, the secretion of IL-6 and TNF-α showed a modest increase with a major decrease in adiponectin secretion (FIGS. 13A–13I). Under hyperglycemia, IL-6, TNF-α, and adiponectin secretion were enhanced but with the same trend as 5.5 mM and 12.5 mM glucose levels. The same trend as adiponectin was found for the calcium and insulin secretion (FIGS. 14A–14F). These findings suggest that in diabetic conditions, insulin secretion from beta cells under hypoxia amplifies TNF-α secretion from adipocytes, which subsequently impairs IL-6’s regulatory function, leading to reduced adiponectin secretion.

In various aspects, the present disclosure contemplates microfluidic devices for regulating flow shear stress on cells. The device may be a microfluidic system featuring two embedded microTesla pumps designed to modulate shear stress over cells, for example, cells co-cultured in chambers under diabetic and hypoxic conditions. The microfluidic device may comprise a gas-permeable membrane disposed beneath a 3D-printed PDMS cover, which has a supply inlet leading into a microfluidic gas chamber and four exhaust/outlets positioned beside the four chambers. A microfluidic channel substrate is optionally placed on top of a slide, and beneath the gas-permeable membrane, with a single microchannel linking the gas supply inlet to the gas exhaust outlets. Gas is introduced via the supply inlet, flowing through the system, with the gas-permeable membrane facilitating gas transfer into the chambers. MicroTesla pumps facilitate continuous flow of a fluid medium within the perfusion chambers. The microTesla pumps may be monitored using a magnetic stirrer to allow the perfusion of flow shear modulation over co-cultured cells. This device allows the modeling of different diseases on-chip while studying the bi-directional interaction under the influence of shear stress, physiologically relevant to blood flow.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A microfluidic device for regulating flow shear stress on cells, the microfluidic device comprising:
a first pair of enclosed perfusion chambers comprising a first perfusion chamber and a second perfusion chamber fluidly coupled with one another;
a second pair of enclosed perfusion chambers comprising a third perfusion chamber and a fourth perfusion chamber fluidly coupled with one another, wherein each of the first, second, third, and fourth perfusion chambers comprises a first inlet for receiving a stream comprising cells, a second inlet for receiving a fluid medium, a first outlet for removing the stream comprising cells, a second outlet through which the fluid medium exits;
a gas permeable membrane having a first surface that defines at least one side of each of the first, second, third, and fourth perfusion chambers;
a first microTesla pump in fluid communication with the second inlet of the first perfusion chamber and the second inlet of the second perfusion chamber configured to circulate the fluid medium therethrough;
a second microTesla pump in fluid communication with the second inlet of the third perfusion chamber and the second inlet of the fourth perfusion chamber configured to circulate the fluid medium therethrough; and
a microfluidic gas channel in fluid communication with and disposed along a second surface of the gas permeable membrane opposite to the first surface, wherein an inlet of the microfluidic gas channel receives a gas that transfers through select regions of the gas permeable membrane into each of the first, second, third, and fourth perfusion chambers.

2. The microfluidic device of claim 1, wherein the first perfusion chamber contains a first type of cells and the second perfusion chamber contains a second type of cells distinct from the first type, the third perfusion chamber contains the first type of cells, and the fourth perfusion chamber contains the second type of cells.

3. The microfluidic device of claim 2, wherein the first type of cells and the second type of cells are independently selected from pancreatic beta cells and adipocytes.

4. The microfluidic device of claim 1, wherein the microfluidic gas channel has a height of greater than or equal to about 0.1 millimeters to less than or equal to about 2 millimeters.

5. The microfluidic device of claim 1, wherein the gas permeable membrane has a thickness of greater than or equal to about 50 micrometers to less than or equal to about 250 micrometers and comprises a porous polydimethylsiloxane (PDMS).

6. The microfluidic device of claim 1, wherein the first inlet and the first outlet are configured to be sealed.

7. The microfluidic device of claim 1, wherein each of the first, second, third, and fourth perfusion chambers has a height of greater than or equal to about 2 millimeters to less than or equal to about 4 millimeters.

8. The microfluidic device of claim 1, wherein the fluid medium continuously flows through the first, second, third, and fourth perfusion chambers during a second operational mode.

9. The microfluidic device of claim 1, wherein the gas comprises oxygen and an oxygen level in each of the first, second, third, and fourth perfusion chambers is independently controlled to be at least one of a hypoxic environment or a normoxic environment.

10. The microfluidic device of claim 1, wherein the first microTesla pump and the first pair of enclosed perfusion chambers define a first recirculation loop, wherein the fluid medium exits the first microTesla pump, is pumped into the second inlet of the first perfusion chamber, exits the second outlet of the first perfusion chamber, enters the second outlet of the second perfusion chamber, exits the second outlet of the second perfusion chamber, and enters the first microTesla pump; and
the second microTesla pump and the second pair of enclosed perfusion chambers define a second recirculation loop, wherein the fluid medium exits the second microTesla pump, is pumped into the second inlet of the third perfusion chamber, exits the second outlet of the third perfusion chamber, enters the second outlet of the fourth perfusion chamber, exits the second outlet of the fourth perfusion chamber, and enters the second microTesla pump.

11. The microfluidic device of claim 1, wherein the cells grow on the first surface of the gas permeable membrane in each of the first, second, third, and fourth perfusion chambers.

12. A method for co-culturing distinct cell types in a microfluidic device under flow shear stress, the method comprising:
pumping a first fluid medium in a first microfluidic recirculation loop comprising a first pair of enclosed perfusion chambers that includes a first perfusion chamber comprising a first type of cells and a second perfusion chamber comprising a second type of cells and a first microTesla pump to induce flow shear stress conditions on the first type of cells in the first perfusion chamber and the second type of cells in the second perfusion chamber;
pumping a second fluid medium in a second microfluidic recirculation loop comprising a second pair of enclosed perfusion chambers that include a third perfusion chamber comprising the first type of cells and a fourth perfusion chamber comprising the second type of cells, and a second microTesla pump to induce flow shear stress conditions on the first type of cells in the third perfusion chamber and the second type of cells in the fourth perfusion chamber; and
introducing gas into a microfluidic gas channel where the gas transfers into each of the first, second, third, and fourth perfusion chambers through a gas permeable membrane, wherein the gas permeable membrane defines at least one wall of the microfluidic chamber.

13. The method of claim 12, wherein the first fluid medium continuously flows through the first and second perfusion chambers during a first operational mode and the second fluid medium continuously flows through the third and fourth perfusion chambers during the first operational mode.

14. The method of claim 12, wherein the pumping of the first fluid medium, pumping of the second fluid medium, and the introducing occur concurrently.

15. The method of claim 12, wherein each of the first, second, third, and fourth perfusion chambers comprises a first inlet for receiving a stream comprising either the first type of cells or the second type of cells, a second inlet for receiving either the first fluid medium or the second fluid medium, a first outlet for removing the stream, and a second outlet through which the first fluid medium or the second fluid medium exits.

16. The method of claim 15, wherein the stream first comprises the first type of cells and the method further comprises passing the stream through the first perfusion chamber and the third perfusion chamber to introduce the first type of cells, and after passing the stream through the first perfusion chamber and the third perfusion chamber, sealing the first outlet of the first perfusion chamber and the third perfusion chamber, and second, the stream comprises the second type of cells and the method further comprises passing the stream through the second perfusion chamber and the fourth perfusion chamber to introduce the second type of cells, and after passing the stream through the second perfusion chamber and the fourth perfusion chamber, sealing the first outlet of the second perfusion chamber and the fourth perfusion chamber.

17. The method of claim 12, wherein the first fluid medium passes through the first microfluidic recirculation loop at a flow rate of greater than or equal to about 0.62 mL/minute to less than or equal to about 1 mL/minute and the second fluid medium passes through the second microfluidic recirculation loop at a flow rate of greater than or equal to about 0.62 mL/minute to less than or equal to about 1 mL/minute.

18. The method of claim 12, further comprising assessing interactions of the first type of cells and the second type of cells under an influence of shear stress related to at least two distinct diseases or conditions.

19. The method of claim 12, where the gas comprises oxygen and is transferred through the gas permeable membrane into each of the first, second, third, and fourth perfusion chambers and an oxygen level in each of the first, second, third, and fourth perfusion chambers is independently controlled to be at least one of a hypoxic environment or a normoxic environment.

20. The method of claim 12, wherein the first type of cells and the second type of cells are independently selected from pancreatic beta cells and adipocytes.
